SEQUENCE LISTING (55 sequences)

SEQ ID NO: 1 - RNA, 42 nt, Artificial sequence
  Synthetic polynucleotide
  misc_feature (1)..(1): 5′ Cy3
  modified_base (1)..(1): um
  modified_base (2)..(3): gm
  misc_feature (4)..(39): ribonucleotide
  modified_base (40)..(40): um
  modified_base (41)..(41): um
uggggggugucuuugggguuguuuuagagcuaugcuguuuug

SEQ ID NO: 2 - RNA, 39 nt, Artificial sequence
  Synthetic polynucleotide
  misc_feature (1)..(1): 5′ Cy3
  modified_base (1)..(1): um
  modified_base (2)..(3): 2′-O methyladenosine
  misc_feature (4)..(36): ribonucleotide
  modified_base (37)..(39): gm
uaauuucuacucuuguagauggguuugguugguucgggg

SEQ ID NO: 3 - RNA, 39 nt, Artificial sequence; identical to SEQ ID NO: 2
  Synthetic polynucleotide
  misc_feature (1)..(36): ribonucleotide
  modified_base (37)..(39): gm
  misc_feature (39)..(39): 3′ Cy3

SEQ ID NO: 4 - RNA, 39 nt, Artificial sequence; identical to SEQ ID NO: 2
  Synthetic polynucleotide
  modified_base (1)..(1): um
  misc_feature (2)..(4): ribonucleotide
  modified_base (5)..(8): um
  modified_base (7)..(7): cm
  modified_base (9)..(9): 2′-O methyladenosine
  modified_base (10)..(10): cm
  misc_feature (11)..(11): ribonucleotide
  modified_base (12)..(12): cm
  modified_base (13)..(14): um
  misc_feature (15)..(15): ribonucleotide
  modified_base (16)..(16): um
  modified_base (18)..(18): gm
  modified_base (19)..(19): 2′-O methyladenosine
  misc_feature (20)..(37): ribonucleotide
  modified_base (38)..(39): gm

SEQ ID NO: 5 - RNA, 41 nt, Artificial sequence
  Synthetic polynucleotide
  modified_base (1)..(1): um
  misc_feature (2)..(4): ribonucleotide
  modified_base (5)..(6): um
  modified_base (7)..(7): cm
  modified_base (8)..(8): um
  modified_base (9)..(9): 2′-O methyladenosine
  modified_base (10)..(10): cm
  modified_base (12)..(12): cm
  modified_base (13)..(14): um
  modified_base (16)..(16): um
  modified_base (17)..(17): 2′-O methyladenosine
  modified_base (18)..(18): gm
  misc_feature (20)..(38): ribonucleotide
  modified_base (39)..(39): gm
  modified_base (40)..(40): um
  modified_base (41)..(41): 2′-O methyladenosine
  misc_feature (41)..(41): 3′ Cy3
uaauuucuacucuuguagauggguuugguugguucggggua

SEQ ID NO: 6 - RNA, 36 nt, Artificial sequence
  Synthetic polynucleotide
  misc_feature (1)..(1): 5′ Cy3
  modified_base (1)..(1): gm
  modified_base (2)..(2): 2′-O methyladenosine
  modified_base (3)..(3): gm
  modified_base (4)..(4): um
  misc_feature (5)..(26): ribonucleotide
  modified_base (27)..(27): gm
  modified_base (28)..(28): 2′-O methyladenosine
  modified_base (29)..(29): gm
  modified_base (30)..(30): cm
  modified_base (31)..(31): um
  modified_base (32)..(32): 2′-O methyladenosine
  modified_base (33)..(33): um
  modified_base (34)..(34): gm
  modified_base (35)..(35): cm
  modified_base (36)..(36): um
gagugguuaguuuuauuaggguuuuagagcuaugcu

SEQ ID NO: 7 - RNA, 67 nt, Artificial sequence
  Synthetic polynucleotide
  modified_base (1)..(1): 2′-O methyladenosine
  misc_feature (1)..(2): phosphorothioate bond
  modified_base (2)..(2): gm
  misc_feature (2)..(3): phosphorothioate bond
  modified_base (3)..(3): cm
  modified_base (4)..(4): 2′-O methyladenosine
  modified_base (5)..(5): um
  modified_base (6)..(6): 2′-O methyladenosine
  modified_base (7)..(7): gm
  modified_base (8)..(8): cm
  modified_base (9)..(9): 2′-O methyladenosine
  modified_base (33)..(33): 2′-O methyladenosine
  modified_base (34)..(34): um
  modified_base (35)..(35): cm
  modified_base (36)..(37): 2′-O methyladenosine
  modified_base (38)..(38): cm
  modified_base (39)..(40): um
  modified_base (41)..(41): gm
  modified_base (42)..(46): 2′-O methyladenosine
  modified_base (47)..(47): gm
  modified_base (48)..(48): um
  modified_base (49)..(50): gm
  modified_base (51)..(51): cm
  modified_base (52)..(52): 2′-O methyladenosine
  modified_base (53)..(54): cm
  modified_base (55)..(55): gm
  modified_base (56)..(56): 2′-O methyladenosine
  modified_base (57)..(57): gm
  modified_base (58)..(58): um
  modified_base (59)..(59): cm
  modified_base (60)..(61): gm
  modified_base (62)..(62): um
  modified_base (64)..(64): cm
  modified_base (65)..(67): um
  misc_feature (65)..(66): phosphorothioate bond
  misc_feature (66)..(67): phosphorothioate bond
agcauagcaaguuaaaauaaggcuaguccguuaucaacuugaaaaaguggcaccgagucggugcuuu

SEQ ID NO: 8 - DNA, 41 bp, Artificial sequence
  Synthetic polynucleotide
  misc_feature (1)..(1): 5′ Cy5
  misc_feature (41)..(41): 3′ Cy3
taatttctactcttgtagatgggtttggttggttcggggta

SEQ ID NO: 9 - RNA, 67 nt, Artificial sequence; identical to SEQ ID NO: 7
  Synthetic polynucleotide
  misc_feature (1)..(1): 5′ Cy5
  modified_base (1)..(1): 2′-O methyladenosine
  modified_base (2)..(2): gm
  modified_base (3)..(3): cm
  modified_base (4)..(4): 2′-O methyladenosine
  modified_base (5)..(5): um
  modified_base (6)..(6): 2′-O methyladenosine
  modified_base (7)..(7): gm
  modified_base (8)..(8): cm
  modified_base (9)..(9): 2′-O methyladenosine
  misc_feature (10)..(37): ribonucleotide
  modified_base (38)..(38): cm
  modified_base (39)..(40): um
  modified_base (41)..(41): gm
  modified_base (42)..(46): 2′-O methyladenosine
  modified_base (47)..(47): gm
  modified_base (48)..(48): um
  modified_base (49)..(50): gm
  modified_base (51)..(51): cm
  modified_base (52)..(52): 2′-O methyladenosine
  modified_base (53)..(54): cm
  modified_base (55)..(55): gm
  modified_base (56)..(56): 2′-O methyladenosine
  modified_base (57)..(57): gm
  modified_base (58)..(58): um
  modified_base (59)..(59): cm
  modified_base (60)..(61): gm
  modified_base (62)..(62): um
  modified_base (63)..(63): gm
  modified_base (64)..(64): cm
  modified_base (65)..(67): um

SEQ ID NO: 10 - RNA, 41 nt, Artificial sequence; identical to SEQ ID NO: 5
  Synthetic polynucleotide
  misc_feature (1)..(1): 5′ Cy5
  modified_base (1)..(1): um
  misc_feature (2)..(4): ribonucleotide
  modified_base (5)..(8): um
  modified_base (7)..(7): cm
  modified_base (9)..(9): 2′-O methyladenosine
  modified_base (10)..(10): cm
  misc_feature (11)..(11): ribonucleotide
  modified_base (12)..(12): cm
  modified_base (13)..(14): um
  misc_feature (15)..(15): ribonucleotide
  modified_base (16)..(16): um
  modified_base (17)..(17): 2′-O methyladenosine
  modified_base (18)..(18): gm
  modified_base (19)..(19): 2′-O methyladenosine
  misc_feature (20)..(38): ribonucleotide
  modified_base (39)..(39): gm
  modified_base (40)..(40): um
  modified_base (41)..(41): 2′-O methyladenosine

SEQ ID NO: 11 - RNA, 56 nt, Artificial sequence
  Synthetic polynucleotide
  misc_feature (1)..(1): 5′ Cy5
  modified_base (1)..(1): gm
  modified_base (2)..(2): um
  modified_base (3)..(3): cm
  modified_base (4)..(7): 2′-O methyladenosine
  modified_base (8)..(8): gm
  modified_base (9)..(9): 2′-O methyladenosine
  modified_base (10)..(11): cm
  modified_base (12)..(16): um
  misc_feature (17)..(19): ribonucleotide
  modified_base (20)..(21): um
  modified_base (22)..(22): cm
  modified_base (23)..(23): um
  modified_base (24)..(24): 2′-O methyladenosine
  modified_base (25)..(25): cm
  misc_feature (26)..(26): ribonucleotide
  modified_base (27)..(27): cm
  modified_base (28)..(29): um
  misc_feature (30)..(30): ribonucleotide
  modified_base (31)..(31): um
  modified_base (32)..(32): 2′-O methyladenosine
  modified_base (33)..(33): gm
  modified_base (34)..(34): 2′-O methyladenosine
  misc_feature (35)..(53): ribonucleotide
  modified_base (54)..(54): gm
  modified_base (55)..(55): um
  modified_base (56)..(56): 2′-O methyladenosine
gucaaaagaccuuuuuaauuucuacucuuguagauggguuugguugguucggggua

SEQ ID NO: 12 - DNA, 41 bp, Artificial sequence; identical to SEQ ID NO: 8
  Synthetic polynucleotide
  misc_feature (1)..(1): 5′ Cy3

SEQ ID NO: 13 - DNA, 41 bp, Artificial sequence; identical to SEQ ID NO: 8
  Synthetic polynucleotide
  misc_feature (41)..(41): 3′ Cy3

SEQ ID NO: 14 - DNA, 67 bp, Artificial sequence
  Synthetic polynucleotide
  misc_feature (1)..(1): 5′ Cy5
agcatagcaagttaaaataaggctagtccgttatcaacttgaaaaagtggcaccgagtcggtgcttt

SEQ ID NO: 15 - DNA, 41 bp, Artificial sequence; identical to SEQ ID NO: 8
  Synthetic polynucleotide
  misc_feature (1)..(1): 5′ ATTO247n

SEQ ID NO: 16 - DNA, 41 bp, Artificial sequence; identical to SEQ ID NO: 8
  Synthetic polynucleotide
  misc_feature (41)..(41): 3′ Cy3

SEQ ID NO: 17 - DNA, 41 bp, Artificial sequence; identical to SEQ ID NO: 8
  Synthetic polynucleotide
  misc_feature (1)..(1): 5′ AmMC6
  misc_feature (41)..(41): 3′ Cy3

SEQ ID NO: 18 - DNA, 41 bp, Artificial sequence; identical to SEQ ID NO: 8
  Synthetic polynucleotide
  misc_feature (1)..(1): 5dSp
  misc_feature (41)..(41): 3′ Cy3

SEQ ID NO: 19 - protein, 1368 aa, Streptococcus pyogenes [sequence omitted]

SEQ ID NO: 20 - DNA, 90 bp, Artificial sequence
  Synthetic polynucleotide
  misc_feature (1)..(1): 5′ Cy5
gctgctaaccccataccccgaaccaaccaaaccccaaagacaatgggcaagcccatccccaaccccttgcttggcttggacagcacctaa

SEQ ID NO: 21 - DNA, 90 bp, Artificial sequence
  Synthetic polynucleotide
  misc_feature (1)..(1): 5′ Cy3
ttaggtgctgtccaagccaagcaaggggttggggatgggcttgcccattgtctttggggtttggttggttcggggtatggggttagcagc

SEQ ID NO: 22 - RNA, 41 nt, Artificial sequence
  Synthetic polynucleotide
  misc_feature (1)..(1): 5′ Cy5
  modified_base (1)..(1): um
  misc_feature (2)..(4): unmodified RNA nucleotide
  modified_base (5)..(6): um
  modified_base (7)..(7): cm
  modified_base (8)..(8): um
  modified_base (10)..(10): cm
  misc_feature (11)..(11): unmodified RNA nucleotide
  modified_base (12)..(12): cm
  modified_base (13)..(14): um
  misc_feature (15)..(15): unmodified RNA nucleotide
  modified_base (18)..(18): gm
  misc_feature (20)..(38): unmodified RNA nucleotide
  modified_base (39)..(41): um
uaauuucuacucuuguagauucuuuuggcgguaugcacuuu

SEQ ID NO: 23 - RNA, 36 nt, Artificial sequence
  Synthetic polynucleotide
  misc_feature (1)..(1): 5′ Cy3
  modified_base (1)..(2): 2′-O-methyladenosine
  modified_base (3)..(4): um
  misc_feature (5)..(26): unmodified RNA nucleotide
  modified_base (27)..(27): gm
  modified_base (28)..(28): 2′-O-methyladenosine
  modified_base (29)..(29): gm
  modified_base (30)..(30): cm
  modified_base (31)..(31): um
  modified_base (32)..(32): 2′-O-methyladenosine
  modified_base (33)..(33): um
  modified_base (34)..(34): gm
  modified_base (35)..(35): cm
  modified_base (36)..(36): um
aauuugaaaucugguuaggcguuuuagagcuaugcu

SEQ ID NO: 24 - protein, 25 aa, Artificial sequence
  Synthetic polypeptide
MLSLRQSIRFFKPATRTLCSSRYLL

SEQ ID NO: 25 - protein, 29 aa, Artificial sequence
  Synthetic polypeptide
MSVLTPLLLRGLTGSARRLPVPRAKIHSL

SEQ ID NO: 26 - DNA, 87 bp, Artificial sequence
  Synthetic polynucleotide
atgtccgtcctgacgccgctgctgctgcggggcttgacaggctcggcccggcggctcccagtgccgcgcgccaagatccattcgttg

SEQ ID NO: 27 - DNA, 34 bp, Artificial sequence
  Synthetic polynucleotide
acctgactgactacctcatgaagatcctcaccga

SEQ ID NO: 28 - DNA, 36 bp, Artificial sequence
  Synthetic polynucleotide
ggagctggaagcagccgtggccatctcttgctcgaa

SEQ ID NO: 29 - DNA, 27 bp, Artificial sequence
  Synthetic polynucleotide
  misc_feature (1)..(1): 5′-Cy5
  misc_feature (27)..(27): black hole quencher
agcgggaaatcgtgcgtgacattaagg

SEQ ID NO: 30 - DNA, 27 bp, Artificial sequence
  Synthetic polynucleotide
gcctatattacggatcatttctctact

SEQ ID NO: 31 - DNA, 24 bp, Artificial sequence
  Synthetic polynucleotide
gcctatgaaggctgttgctatagt

SEQ ID NO: 32 - DNA, 28 bp, Artificial sequence
  Synthetic polynucleotide
  misc_feature (1)..(1): 5′-HEX
  misc_feature (28)..(28): black hole quencher
cctgaaacatcggcattatcctcctgct

SEQ ID NO: 33 - DNA, 22 bp, Artificial sequence
  Synthetic polynucleotide
ggcccgttgtcaggcaacgtgg

SEQ ID NO: 34 - DNA, 28 bp, Artificial sequence
  Synthetic polynucleotide
ggaaaggagctgacaggtggtggcaatg

SEQ ID NO: 35 - DNA, 22 bp, Artificial sequence
  Synthetic polynucleotide
  misc_feature (1)..(1): 5′-FAM
  misc_feature (22)..(22): black hole quencher
tgctgacgcaacccccactggt

SEQ ID NO: 36 - RNA, 36 nt, Artificial sequence
  Synthetic polynucleotide
  misc_feature (1)..(1): 5′Cy3
  modified_base (1)..(1): um
  modified_base (2)..(4): 2′-O-Methyladenosine
  misc_feature (5)..(26): ribonucleotide
  modified_base (27)..(27): gm
  modified_base (28)..(28): 2′-O-Methyladenosine
  modified_base (29)..(29): gm
  modified_base (30)..(30): cm
  modified_base (31)..(31): um
  modified_base (32)..(32): 2′-O-Methyladenosine
  modified_base (33)..(33): um
  modified_base (34)..(34): gm
  modified_base (35)..(35): cm
  modified_base (36)..(36): um
uaaacuguggggggugucuuguuuuagagcuaugcu

SEQ ID NO: 37 - RNA, 36 nt, Artificial sequence
  Synthetic polynucleotide
  misc_feature (1)..(1): 5′Cy3
  modified_base (1)..(1): gm
  modified_base (2)..(3): 2′-O-Methyladenosine
  modified_base (4)..(4): gm
  misc_feature (5)..(26): ribonucleotide
  modified_base (27)..(27): gm
  modified_base (28)..(28): 2′-O-Methyladenosine
  modified_base (29)..(29): gm
  modified_base (30)..(30): cm
  modified_base (31)..(31): um
  modified_base (32)..(32): 2′-O-Methyladenosine
  modified_base (33)..(33): um
  modified_base (34)..(34): gm
  modified_base (35)..(35): cm
  modified_base (36)..(36): um
gaagcgugaugacaaagaggguuuuagagcuaugcu

SEQ ID NO: 38 - RNA, 41 nt, Artificial sequence
  Synthetic polynucleotide
  misc_feature (1)..(1): 5′Cy5
  modified_base (1)..(1): um
  misc_feature (2)..(4): ribonucleotide
  modified_base (5)..(8): um
  modified_base (7)..(7): cm
  modified_base (9)..(9): 2′-O-Methyladenosine
  modified_base (10)..(10): cm
  misc_feature (11)..(11): ribonucleotide
  modified_base (12)..(12): cm
  modified_base (13)..(14): um
  misc_feature (15)..(15): ribonucleotide
  modified_base (16)..(16): um
  modified_base (17)..(17): 2′-O-Methyladenosine
  modified_base (18)..(18): gm
  modified_base (19)..(19): 2′-O-Methyladenosine
  misc_feature (20)..(38): ribonucleotide
  modified_base (39)..(40): 2′-O-Methyladenosine
  modified_base (41)..(41): cm
uaauuucuacucuuguagaucagauauacacuucagauaac

SEQ ID NO: 39 - RNA, 41 nt, Artificial sequence; identical to SEQ ID NO: 5
  Synthetic polynucleotide
  misc_feature (1)..(1): 5′Cy5
  modified_base (1)..(1): um
  misc_feature (2)..(4): ribonucleotide
  modified_base (5)..(8): um
  modified_base (5)..(6): um
  modified_base (7)..(7): cm
  modified_base (8)..(8): um
  modified_base (9)..(9): 2′-O-Methyladenosine
  modified_base (10)..(10): cm
  misc_feature (11)..(11): ribonucleotide
  modified_base (12)..(12): cm
  modified_base (13)..(14): um
  misc_feature (15)..(15): ribonucleotide
  modified_base (16)..(16): um
  modified_base (17)..(17): 2′-O-Methyladenosine
  modified_base (18)..(18): gm
  modified_base (19)..(19): 2′-O-Methyladenosine
  misc_feature (20)..(38): ribonucleotide
  modified_base (39)..(39): gm
  modified_base (40)..(40): um
  modified_base (41)..(41): 2′-O-Methyladenosine

SEQ ID NO: 40 - DNA, 21 bp, Artificial sequence
  Synthetic polynucleotide
gcacagccactttccacacag

SEQ ID NO: 41 - DNA, 23 bp, Artificial sequence
  Synthetic polynucleotide
gggagtgggaggggaaaataatg

SEQ ID NO: 42 - DNA, 28 bp, Artificial sequence
  Synthetic polynucleotide
  misc_feature (1)..(1): 5′-FAM
  misc_feature (28)..(28): black hole quencher
caaagaaccctaacaccagcctaaccag

SEQ ID NO: 43 - DNA, 23 bp, Artificial sequence
  Synthetic polynucleotide
catcctacccagcacacacacac

SEQ ID NO: 44 - DNA, 23 bp, Artificial sequence
  Synthetic polynucleotide
cggggatgcttgcatgtgtaatc

SEQ ID NO: 45 - DNA, 27 bp, Artificial sequence
  Synthetic polynucleotide
  misc_feature (1)..(1): 5′-FAM
  misc_feature (27)..(27): black hole quencher
caaccaaaccccaaagacaccccccac

SEQ ID NO: 46 - protein, 3 aa, Artificial sequence
  Synthetic polypeptide
NGG

SEQ ID NO: 47 - protein, 5 aa, Artificial sequence
  Synthetic polypeptide
NGRRT

SEQ ID NO: 48 - protein, 5 aa, Artificial sequence
  Synthetic polypeptide
NGRRN

SEQ ID NO: 49 - protein, 8 aa, Artificial sequence
  Synthetic polypeptide
NNNNGATT

SEQ ID NO: 50 - protein, 7 aa, Artificial sequence
  Synthetic polypeptide
NNAGAAW

SEQ ID NO: 51 - protein, 6 aa, Artificial sequence
  Synthetic polypeptide
NAAAAC

SEQ ID NO: 52 - protein, 3 aa, Artificial sequence
  Synthetic polypeptide
TTN

SEQ ID NO: 53 - protein, 3 aa, Artificial sequence
  Synthetic polypeptide
NGG

SEQ ID NO: 54 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPEFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFIKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNFGAPAAFIYFDTTIGRNRYKSIKEVLDATLIHQSITGLYETRIDLSQLGGD

SEQ ID NO: 55 - protein, 1388 aa, Streptococcus pyogenes
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

(19) United States
(12) Patent Application Publication
(10) Pub. No.: US 2021/0310002 A1
(43) Pub. Date: Oct. 7, 2021

(54) CATIONIC COMPOUNDS FOR DELIVERY OF NUCLEIC ACIDS

(71) Applicants: City of Hope, Duarte, CA (US); The Regents of the University of California, Oakland, CA (US)

(72) Inventors: John BURNETT, Chatsworth, CA (US); Anh PHAM, Chatsworth, CA (US)

(21) Appl. No.: 17/270,832

(22) PCT Filed: Aug. 30, 2019
(86) PCT No.: PCT/US2019/049165

§ 371 Date: Feb. 23, 2021

Related U.S. Application Data
(60) Provisional application No. 62/726,004, filed on Aug. 31, 2018.

(51) Int. Cl. C12N 15/113; A61K 31/712; A61K 47/54; C12N 15/90

(52) U.S. Cl. CPC C12N 15/113; A61K 31/712; A61K 47/545; C12N 2320/32; C12N 2310/11; C12N 2310/315; C12N 2310/3517; C12N 15/90

(57) ABSTRACT

Provided herein, inter alia, are compositions, including compounds comprising a polynucleotide covalently linked to a cyanine moiety. In embodiments, polynucleotides comprise a nucleotide sequence that is fully complementary to a nucleotide sequence of a mitochondrial polynucleotide. Methods of using such compounds are also provided, including for targeting mitochondrial DNA, such as for cleaving, modifying, or altering the expression of mitochondrial DNA.

### CROSS-REFERENCE

This application claims the benefit of priority to U.S. Provisional Patent Application Ser. No. 62/726,004, filed Aug. 31, 2018, which is hereby incorporated by reference in its entirety for all purposes.

### SEQUENCE LISTING

The material in the accompanying Sequence Listing is hereby incorporated by reference in its entirety. The accompanying Sequence Listing file, named “048440-680001WO_SL_ST25”, was created on Aug. 29, 2019 and is 66,208 bytes in size.

### BACKGROUND OF THE INVENTION

Mitochondria are unique dynamic organelles that provide energy for the cell in the form of ATP and carry genomic content. Mitochondrial DNA (mtDNA) encodes for critical subunits in the electron transport chain, and mutations in mtDNA have devastating bioenergetic defects resulting in, for example, neuromuscular diseases. Gene therapy approaches aimed at correcting mutated genes have been limited by the challenges of transforming mtDNA. There is a great demand for methods to access mtDNA for purposes of performing gene therapy. Provided herein, inter alia, are solutions to these and other problems in the art.

### BRIEF SUMMARY OF THE INVENTION

Provided herein are, inter alia, compounds, complexes, and compositions comprising a cyanine moiety conjugated to a polynucleotide. Also included are methods for modifying and altering the expression of mitochondrial DNA and RNA molecules.

In an aspect, included herein is a compound comprising a polynucleotide covalently linked to a cyanine moiety, wherein the polynucleotide comprises a nucleotide sequence that is fully complementary to a nucleotide sequence of a mitochondrial polynucleotide.

In an aspect, included herein is a compound comprising a polynucleotide covalently linked to a cyanine moiety, wherein the cyanine moiety is attached at the 5′-end of the polynucleotide, and wherein the polynucleotide comprises one or more ribonucleotides.

In an aspect, included herein is cell comprising a compound or complex disclosed herein.

In an aspect, included herein is a complex comprising a protein and a compound disclosed herein. In embodiments, the protein is an RNA-guided protein.

In an aspect, included herein is a method of reducing the expression of a mitochondrial protein and/or polynucleotide.

In an aspect, included herein is a method of altering the sequence of a mitochondrial polynucleotide (e.g., DNA).

In an aspect, included herein is a method of altering the sequence or the expression of at least one mitochondrial polynucleotide. In embodiments, the method comprises introducing into a eukaryotic cell an effective amount of a compound or complex described herein.

In an aspect, included herein is a method of treating a mitochondrial disorder in a subject in need thereof. In embodiments, the method comprises administering to the subject an effective amount of a compound or complex described herein.

### BRIEF DESCRIPTION OF THE DRAWINGS

FIGS. 1A-1B. Mitochondrial localization of Cpf1 from Acidaminococcus sp. (“AsCpf1,” also known as AsCas12a). (FIG. 1A) Schematic of plasmid construct for targeting AsCpf1 to mitochondria using Cox8 mitochondrial targeting signal. (FIG. 1B) Images showing colocalization of HA-tagged AsCpf1.

FIGS. 2A-2B. Delivery of single-stranded (ss) and double-stranded (ds) DNA oligonucleotide to the mitochondria. (FIG. 2A) A single-stranded DNA oligonucleotides 91 nt in length was labeled on the 5′ end with Cy5, while the fully complementary single-stranded DNA 91 nt oligonucleotide was labeled on the 5′ end with Cy3. The sense (Cy5-labeled; top row) and antisense (Cy3-labeled; middle row) oligos each localized efficiently to mitochondria when transfected alone, as determined by co-localization with Mitotracker Green. Co-transfection of both sense (Cy5-labeled) and antisense (C3-labeled) oligos resulted in co-localization of Cy5 and Cy3 signals with Mitotracker Green, indicating efficient mitochondrial import of linear double-stranded DNA (last row of FIG. 2A; sense, antisense, mitotracker, and merged, from left to right). Pre-transfection hybridization of complementary ssDNA oligonucleotides to form a long duplex did not impair import into the mitochondria (last row of FIG. 2A). (FIG. 2B) Quantification of the Mander's correlation coefficient for the ssDNA or dsDNA oligonucleotide depicted in FIG. 2A (legend from top to bottom corresponding to bars from left to right in each triplet). The Mander's correlation coefficients were comparable for both the ssDNA and dsDNA versions suggesting a similar import efficiency and mechanism. Scale bar represents 10 μm.

FIGS. 3A-3B. Persistence of Cy3 crRNA in mitochondria. (FIG. 3A) Schematic of AsCpf1 crRNA molecule targeted to mitochondria. The Cy3 dye is located at the 5′ end and asterisks represent 2′OMe modifications of 3 nucleotides at both ends of the RNA. (FIG. 3B) Fluorescence micrographs demonstrating stable Cy3 RNA signal in mitochondria even after 48 h post transfection by streptolysin O (SLO). Scale bar is 10 μm.

FIGS. 4A-4D. Cryofixation followed by transmission electron microscopy (TEM) of DNA and RNA oligos within the mitochondrial matrix. (FIG. 4A) and (FIG. 4B) are representative images of DNA oligos immunostained with a sheep anti-Cy3 antibody followed by a 6 nm gold conjugated anti-sheep secondary antibody. Half arrows highlight the gold particles. (FIG. 4C) and (FIG. 4D) are representative images of the RNA oligos demonstrating matrix localization. Scale bars are 200 nm.

FIGS. 5A-5C. Functional type II CRISPR RNA in the mitochondria. (FIG. 5A) shows fluorescence micrographs of the crRNA and tracrRNA of Cas9 co-localized with Mitotracker Green. Insets highlight the zoomed image of mitochondrial network. Scale bar represents 10 μm. (FIG. 5B) Quantification of co-localization between the RNA signal and the mitochondrial signal using Mander's thresholded correlation coefficient. The tM1 represents RNA signal that co-localizes with the mitochondrial network while the tM2 coefficient shows homogeneity of mitochondrial network containing RNA oligos. (Error bars represent mean±standard deviation, N=20-30 images) Mitochondrial RNA localization is less efficient for the longer tracrRNA. (FIG. 5C) In vitro cutting assay of Cas9 CRISPR system using modified crRNA and tracrRNA. Modifications to crRNA and tracrRNA for mitochondrial import do not affect DNA cleavage by Cas9 endonuclease.

FIGS. 6A-6C. Type V CRISPR RNA in mitochondria. (FIG. 6A) Fluorescence micrographs highlighting the mitochondrial localization of the Cpf1 crRNA at various lengths. Insets show zoomed images of the RNA signal within mitochondrial network. Scale bar is 10 μm. (FIG. 6B) Quantification of co-localization of crRNA with the mitochondrial network using Mander's correlation coefficient. The tM1 value represents the fraction of crRNA co-localized with mitochondria while the tM2 reveal the population of mitochondrial network harboring crRNA. Graph depicts mean value ±standard deviation from N=20-30 images quantified. Note the decreased efficiency of mitochondrial import for longer RNAs. (FIG. 6C) In vitro cutting assay for Cpf1 endonuclease (1 μM) with the modified crRNA (5 μM) and 18 nM of target dsDNA. The 39 nt and 41 nt crRNA versions are effective for Cpf1-mediated cleavage but the pre-processed crRNA version is not functional.

FIGS. 7A-7D. Illustrative effects of 5′ labeling of cyanine dyes on RNA import in mitochondria. (FIGS. 7A-7C) Fluorescence micrographs of various oligos with 5′ and/or 3′ labeling of cyanine dyes. The 5′ labeling mediates successful co-localization of the oligos with the mitochondrial network as seen by Mitotracker Green (FIG. 7A and FIG. 7C). Red circles denote Cy3 dye while blue circles depict Cy5 labeling. The 3′ labeling results in vesicular signal that do not co-localize with mitochondria (FIG. 7B). (FIG. 7D) Quantification of co-localization of oligos with mitochondria by Mander's correlation coefficient. The tM1 and tM2 values are statistically different between the oligos with a 5′ label compared to the oligos with only a 3′ label, * p<0.05 by ANOVA.

FIG. 8. Effects of charge on RNA localization to mitochondria. The addition of neutral 2′ O-methyl enables mitochondrial localization of the RNA (Row A). In contrast, when negatively charged moieties including phosphorothioate (Row B) or 2′ fluoro (Row C) are added to the RNA, most of the oligonucleotides are localized to vesicles that do not colocalize with the mitochondrial marker, Mitotracker green. Scale bar represents 10 μm.

FIGS. 9A-9C. Improved mitochondrial localization with 2′ O-methyl (2′-OMe) modifications of RNA. (A) Fluorescence micrographs of 5′ Cy3 labeled Cas9 crRNA (36 nt) with 39% of nucleotides modified with 2′-OMe. There is mitochondrial colocalization between the labeled RNA and Mitotracker Green. (B) Fluorescence micrographs of another Cas9 crRNA with only 14% 2′-OMe modifications exhibiting only vesicular localization. (C) Quantitation depicting the Mander's correlation coefficient for the respective Cas9 crRNA in panels A and B.

FIG. 10. Illustration of mitochondrial import of RNA affected by mitochondrial membrane potential and independent of Voltage-dependent Anion Channel (VDAC). Dissipation of the mitochondrial membrane potential by treating the cells with carbonyl cyanide m-chlorophenyl hydrazine (CCCP) 40 μM for 30 minutes (Row B) reversed the RNA accumulation in mitochondria compared to the DMSO control (Row A). Most RNA signal redistributed into vesicles that did not colocalize with mitoEGFP, a transfection marker. VDAC exhibited a prominent role in the mitochondrial permeability transition pore (MPTP) that allows molecules to translocate across the mitochondrial membranes. The inhibition of VDAC oligomerization using 4,4′-Diisothiocyanatostilbene-2,2′-disulfonic acid 500 μM (DIDS) did not block RNA import into the mitochondria (Row C), suggesting that the mechanism of oligonucleotide import is independent of the MPTP. Scale bar represents 10 μm.

FIG. 11. Depletion of wild-type mtDNA in HeLa cells using mtCas9. (Panel A) Quantitation of mtDNA content showing depletion of mtDNA in all samples with the crRNA and tracrRNA. Values represent mean±SD from 3 biological replicates. (Panel B) Table of values graphed in Panel A.

FIG. 12. Depletion of mtDNA using mitoCpf1. (Panel A) A graph illustrating that targeting the HSP sequence yielded the highest depletion of mtDNA (left and right bars in each pair represent day 3 and day 5, respectively). (Panel B) Table of values graphed in Panel A. Values represent mean±SD from 3 biological replicates.

FIG. 13. Effects of polynucleotide charge on efficiency of mitochondrial import. (Panel A) Three polynucleotide of similar length and sequences but different charges based on DNA ribose, 2′-OMe modification, or phosphorothioate backbone. (Panel B) The 190 sequence is theorized to be the most negatively charge based on the 4 PS residues, and exhibited the lowest Mander's correlation coefficients. In contrast, the DNA sequence and the RNA sequence with 58% 2′-OMe modifications had the highest colocalization with mitochondria. (Panel C) A 2D plot of Mander's M2 vs M1 for the polynucleotides listed in Panel A.

FIG. 14. Effects of 5′ linkage of Cy dye and 2′-OMe modification on RNA import into mitochondria. (Panel A) Table listing the length, direction of Cy label, and number of 2′-OMe modifications. (Panel B) Graph illustrating Mander's correlation coefficients, M1 and M2, for the listed RNA in the table of Panel A. RNA sequences with a Cy3 or Cy5 moiety at the 5′ end of the RNA oligonucleotide (polynucleotides 171 and 196) exhibited efficient mitochondrial localization. The extent of 2′-OMe modification did not improve mitochondrial localization when the Cy moiety was attached at the 3′ end of the oligonucleotide. (Panel C) A 2D plot of M2 vs M1 for the RNA listed in Panel A.

FIG. 15. Illustrates structures of example cyanine moieties.

FIG. 16. Illustrates examples of covalent linkages between a cyanine moiety and an oligonucleotide.

FIG. 17. Illustrates an example of a covalent linkage between a cyanine moiety and an oligonucleotide.

FIG. 18. Illustrates structures of modifications that did not efficiently direct transport to the mitochondria.

FIG. 19. Shows mitochondrial localization of Cy5-labeled RNA oligonucleotide in 143B human osteosarcoma cell line. The scale bar is 10 micrometers.

FIG. 20. Shows mitochondrial localization of Cy3-labeled RNA oligonucleotide in human primary T cells. The scale bar is 2 micrometers.

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless specifically defined otherwise, all technical and scientific terms used herein shall be taken to have the same meaning as commonly understood by one of ordinary skill in the art (e.g., in cell culture, molecular genetics, and biochemistry).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

It is understood that where a parameter range is provided, all integers within that range, and tenths thereof, are also provided by the invention. For example, “0.2-5%” is a disclosure of 0.2%, 0.3%, 0.4%, 0.5%, 0.6% etc. up to and including 5.0%.

As used herein, the singular forms “a,” “an,” and “the” include the plural reference unless the context clearly dictates otherwise. Thus, for example, a reference to “a compound,” “a polynucleotide”, or “a cell” is a reference to one or more such embodiments, and includes equivalents thereof known to those skilled in the art and so forth.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Contacting” is used in accordance with its plain ordinary meaning and refers to the process of allowing at least two distinct species (e.g. chemical compounds including biomolecules or cells) to become sufficiently proximal to react, interact or physically touch.

In embodiments, a “patient” or “subject in need thereof” refers to a living member of the animal kingdom who has or that may have or develop (e.g., is at risk of or is suspected of suffering from) the indicated disorder or disease. In embodiments, a subject or patient is a member of a species that includes individuals who naturally suffer from the disorder or disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human. In embodiments, the subject is a non-mammalian animal such as a turkey, a duck, or a chicken. In embodiments, a subject is a living organism suffering from or prone to a disease or condition that can be treated by administration of a compound, complex, or composition as provided herein. The terms “subject,” “patient,” “individual,” etc. can be generally interchanged. In embodiments, an individual described as a “patient” does not necessarily have a given disease or disorder, but may, e.g., be merely seeking medical advice.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” may refer to a decrease in the occurrence of disease symptoms in a patient. As indicated above, the prevention may be complete (no detectable symptoms) or partial, such that fewer symptoms are observed than would likely occur absent treatment.

A “effective amount” is an amount sufficient for a compound to accomplish a stated purpose relative to the absence of the compound (e.g. achieve the effect for which it is administered, treat a disease, modify a polynucleotide, reduce expression, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In embodiments, for any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. In embodiments, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. In embodiments, treatment is initiated with smaller dosages which are less than the optimum dose of the compound. In embodiments, the dosage is increased by small increments until the optimum effect under circumstances is reached. In embodiments, dosage amounts and intervals can be adjusted individually to provide levels of the administered compound effective for the particular clinical indication being treated. In embodiments, this will provide a therapeutic regimen that is commensurate with the severity of the individual's disease state.

The term “administering” includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. In embodiments, administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. In embodiments, the compounds provided herein can be administered alone or can be coadministered to the patient. Coadministration is meant to include simultaneous or sequential administration of the compounds individually or in combination (more than one compound). In embodiments, the preparations can also be combined, when desired, with other active substances (e.g. to reduce metabolic degradation).

An “isolated” or “purified” nucleic acid molecule, polynucleotide, complex, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. In embodiments, a purified compound is one that is at least 90%, 91%, 92%, 93%, 94%, 95%, 98%, 99%, or 100% (w/w) of the desired compound by weight. Purity may be measured by, e.g., any appropriate standard method, for example, by column chromatography, thin layer chromatography, or high-performance liquid chromatography (HPLC) analysis. In embodiments, a purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents. A protein that is the predominant species present in a preparation is substantially purified.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides (e.g. at least two nucleotides) covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer.

Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

An “antisense nucleic acid” as referred to herein is a polynucleotide that is complementary to at least a portion of a specific target nucleic acid (e.g., mitochondrial DNA or RNA) and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). In embodiments, synthetic antisense nucleic acids (e.g. oligonucleotides) are between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbone-modified nucleotides.

In embodiments, in a cell, the antisense nucleic acids hybridize to the corresponding RNA forming a double-stranded molecule. In embodiments, the antisense nucleic acids interfere with the endogenous behavior of the RNA and inhibit its function relative to the absence of the antisense nucleic acid. In embodiments, the double-stranded molecule may be degraded via the RNAi pathway. The use of antisense methods to inhibit the in vitro translation of genes is well known in the art (Marcus-Sakura, Anal. Biochem., 172:289, (1988)). In embodiments, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (e.g., about 60% identity, preferably 65%, 70%, 75%, 80%, 85%, 90%, 91%, 92%, 93%, 94%, 95%, 96%, 97%, 98%, 99%, or higher identity such as 100% over a specified region). In embodiments, a sequence that is complementary (e.g., fully complementary) to a reference sequence (e.g., a mitochondrial polynucleotide) is about or more than about 10, 15, 20, 25, 30, 35, 40, 45, 50, 55, 60, 65, 70, 75, 80, 85, 90, 100, 110, 120, 130, 140, 150, or more nucleotides in length. In embodiments, a sequence that is complementary to a reference sequence is between about 10-150, 25-100, 35-100, or 40-70 nucleotides in length.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —WC(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of 0, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

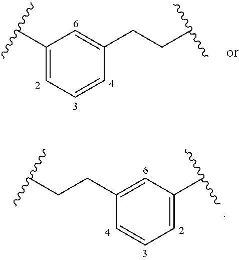 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SW, -halogen, —SiR′R″R′″, —OC(O)R′, —C(O)R′, —CO₂R″, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, SO₄H, —SO₂NH₂, —NHNH₂, —ONH₂, —NHC(O)NHNH₂,
—NHC(O)NH₂, —NHSO₂H, —NHC(O)H,
—NHC(O)OH, —NHOH, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
   —NHC(O)NH₂, —NHSO₂H, —NHC(O)H,
   —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CF₃, —CI₃, CHCl₂, —CHBr₂, —CHF₂, CH₂Cl, —CH₂Br, —CH₂F, —CN, —OH, —NH₂, —COOH, —CONH₂, —NO₂, —SH, —SO₃ H, —SO₂NH₂, —NHNH₂, —ONH₂, —NHC(O)NHNH₂,
      —NHC(O)NH₂, —NHSO₂H, —NHC(O)H,
      —NHC(O)OH, —NHOH, —OCCl₃, —OCF₃, —OCBr₃, —OC₁₃, —OCHCl₂, —OCHBr₂, —OCHF₂, —OCH₂Cl, —OCH₂Br, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, CHCl₂, —CHBr₂, —CHF₂, —
      CH₂Cl, —CH₂Br, —CH₂F, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H,
      —NHC(O)OH, —NHOH, —OCCl₃, —OCF₃, —OCBr₃, —OCI₃, —OCHCl₂, —OCHBr₂, —OCHF₂, —OCH₂Cl, —OCH₂Br, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally differently.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

In general, the term “cyanine dye” refers to a family of polymethine dyes, in which two nitrogens are joined by a polymethine chain. Categories of cyanine dyes include streptocyanines, hemicyanines, and closed cyanines. In embodiments, the cyanine dye includes two indolyl or benzoxazole ring systems interconnected by a conjugated polyene linker. Some particular examples of cyanine dyes include, but are not limited to, the Cy® family of dyes, which include, for example, Cy2, Cy3, Cy3B, Cy3.5, Cy5, Cy5.5, Cy7, Cy9, and derivatives thereof. The term “cyanine moiety”, as used herein, generally refers to a monovalent form of a cyanine dye. In embodiments, a cyanine moiety is conjugated to a polynucleotide. Methods and reagents for conjugating cyanine moieties to polynucleotides are known in the art. Additional examples of cyanine dyes and methods for attachment to polynucleotides can be found in, e.g., US20180258099A1, US20040203038A1, and U.S. Pat. No. 6,110,630.

Each embodiment disclosed herein is contemplated as being applicable to each of the other disclosed embodiments. Thus, all combinations of the various elements described herein are within the scope of the invention.

### II. COMPOUNDS, COMPLEXES, AND COMPOSITIONS

In an aspect, included herein is a compound comprising a polynucleotide covalently linked to a cyanine moiety, wherein the polynucleotide comprises a nucleotide sequence that is fully complementary to a nucleotide sequence of a mitochondrial polynucleotide.

In an aspect, included herein is a compound comprising a polynucleotide covalently linked to a cyanine moiety, wherein the cyanine moiety is attached at the 5′-end of the polynucleotide, and wherein the polynucleotide comprises one or more ribonucleotides.

In embodiments, the mitochondrial polynucleotide is a mitochondrial DNA or a mitochondrial RNA.

In embodiments, the mitochondrial polynucleotide is a mitochondrial ribosomal RNA. In embodiments, the mitochondrial polynucleotide encodes a mitochondrial ribosomal RNA. In embodiments, the mitochondrial polynucleotide is a mitochondrial transfer RNA. In embodiments, the mitochondrial polynucleotide encodes a mitochondrial transfer RNA. In embodiments, the mitochondrial polynucleotide is DNA that encodes a subunit of the respiratory chain (e.g., within complex I, III, IV, or V). In embodiments, the mitochondrial polynucleotide is an mRNA that encodes a subunit of the respiratory chain (e.g., within complex I, III, IV, or V).

In embodiments, the polynucleotide comprises one or more ribonucleotides, one or more deoxyribonucleotides, and/or one or more 2′-modified nucleotides. In embodiments, the one or more 2′-modified nucleotides are 2′-amine modified nucleotides, 2′-O-methyl modified nucleotides or any combination thereof. In embodiments, the polynucleotide comprises any combination of (i) 1, 2, 3, 4, 5, 6, 7, 8, 9, 10, 11, 12, 13, 14, 15, 16, 17, 18, 19, 20, 21, 22, 23, 24, 25, 1-25, 25-50 or 1-50 ribonucleotides; (ii) 1, 2, 3, 4, 5, 6, 7, 8, 9, 10, 11, 12, 13, 14, 15, 16, 17, 18, 19, 20, 21, 22, 23, 24, 25, 1-25, 25-50 or 1-50 deoxyribonucleotides; and/or (iii) 1, 2, 3, 4, 5, 6, 7, 8, 9, 10, 11, 12, 13, 14, 15, 16, 17, 18, 19, 20, 21, 22, 23, 24, 25, 1-25, 25-50 or 1-50 2′-modified nucleotides. the polynucleotide comprises any combination of (i) about or more than about 1%, 5%, 10%, 20%, 25%, 30%, 35%, 40%, 45%, 50%, 60%, 70%, 80%, 90%, 95%, or 100%, or between about 1-100%, 20-80%, or 40-60% ribonucleotides; (ii) about or more than about 1%, 5%, 10%, 20%, 25%, 30%, 35%, 40%, 45%, 50%, 60%, 70%, 80%, 90%, 95%, or 100%, or between about 1-100%, 20-80%, or 40-60% deoxyribonucleotides; and/or (iii) about or more than about 1%, 5%, 10%, 20%, 25%, 30%, 35%, 40%, 45%, 50%, 60%, 70%, 80%, 90%, 95%, or 100%, or between about 1-100%, 20-80%, or 40-60% modified nucleotides.

In embodiments, the polynucleotide comprises one or more ribonucleotides. In embodiments, the polynucleotide comprises 1, 2, 3, 4, 5, 6, 7, 8, 9, 10, 11, 12, 13, 14, 15, 16, 17, 18, 19, 20, 21, 22, 23, 24, 25, 50, 75, 100, or more ribonucleotides. In embodiments, the polynucleotide comprises 1-25, 25-50 or 1-50 ribonucleotides. In embodiments, the polynucleotide comprises 15 ribonucleotides. In embodiments, the polynucleotide comprises 20 ribonucleotides. In embodiments, the polynucleotide comprises 25 ribonucleotides. In embodiments, the polynucleotide comprises 30 ribonucleotides. In embodiments, the polynucleotide comprises 35 ribonucleotides. In embodiments, the polynucleotide comprises 40 ribonucleotides. In embodiments, the polynucleotide comprises 45 ribonucleotides. In embodiments, the polynucleotide comprises 50 ribonucleotides. In embodiments, the polynucleotide comprises 60 ribonucleotides. In embodiments, the polynucleotide comprises 70 ribonucleotides. In embodiments, the polynucleotide comprises 80 ribonucleotides. In embodiments, the polynucleotide comprises 90 ribonucleotides. In embodiments, the polynucleotide comprises 1-100 ribonucleotides. In embodiments, the polynucleotide comprises 10-75 ribonucleotides. In embodiments, the polynucleotide comprises 25-50 ribonucleotides.

In embodiments, about 10% or more of the nucleotides in the polynucleotide are ribonucleotides. In embodiments, about 20% or more of the nucleotides in the polynucleotide are ribonucleotides. In embodiments, about 30% or more of the nucleotides in the polynucleotide are ribonucleotides. In embodiments, about 40% or more of the nucleotides in the polynucleotide are ribonucleotides. In embodiments, about 50% or more of the nucleotides in the polynucleotide are ribonucleotides. In embodiments, about 60% or more of the nucleotides in the polynucleotide are ribonucleotides. In embodiments, about 70% or more of the nucleotides in the polynucleotide are ribonucleotides. In embodiments, about 80% or more of the nucleotides in the polynucleotide are ribonucleotides. In embodiments, about 90% or more of the nucleotides in the polynucleotide are ribonucleotides. In embodiments, all of the nucleotides in the polynucleotide are ribonucleotides. In embodiments, none of the nucleotides in the polynucleotide are ribonucleotides.

In embodiments, the polynucleotide comprises one or more deoxyribonucleotides. In embodiments, the polynucleotide comprises 1, 2, 3, 4, 5, 6, 7, 8, 9, 10, 11, 12, 13, 14, 15, 16, 17, 18, 19, 20, 21, 22, 23, 24, 25, 50, 75, 100, or more deoxyribonucleotides. In embodiments, the polynucleotide comprises 1-25, 25-50 or 1-50 deoxyribonucleotides. In embodiments, the polynucleotide comprises 15 deoxyribonucleotides. In embodiments, the polynucleotide comprises 20 deoxyribonucleotides. In embodiments, the polynucleotide comprises 25 deoxyribonucleotides. In embodiments, the polynucleotide comprises 30 deoxyribonucleotides. In embodiments, the polynucleotide comprises 35 deoxyribonucleotides. In embodiments, the polynucleotide comprises 40 deoxyribonucleotides. In embodiments, the polynucleotide comprises 45 deoxyribonucleotides. In embodiments, the polynucleotide comprises 50 deoxyribonucleotides. In embodiments, the polynucleotide comprises 60 deoxyribonucleotides. In embodiments, the polynucleotide comprises 70 deoxyribonucleotides. In embodiments, the polynucleotide comprises 80 deoxyribonucleotides. In embodiments, the polynucleotide comprises 90 deoxyribonucleotides. In embodiments, the polynucleotide comprises 1-100 deoxyribonucleotides. In embodiments, the polynucleotide comprises 10-75 deoxyribonucleotides. In embodiments, the polynucleotide comprises 25-50 deoxyribonucleotides.

In embodiments, about 10% or more of the nucleotides in the polynucleotide are deoxyribonucleotides. In embodiments, about 20% or more of the nucleotides in the polynucleotide are deoxyribonucleotides. In embodiments, about 30% or more of the nucleotides in the polynucleotide are deoxyribonucleotides. In embodiments, about 40% or more of the nucleotides in the polynucleotide are deoxyribonucleotides. In embodiments, about 50% or more of the nucleotides in the polynucleotide are deoxyribonucleotides. In embodiments, about 60% or more of the nucleotides in the polynucleotide are deoxyribonucleotides. In embodiments, about 70% or more of the nucleotides in the polynucleotide are deoxyribonucleotides. In embodiments, about 80% or more of the nucleotides in the polynucleotide are deoxyribonucleotides. In embodiments, about 90% or more of the nucleotides in the polynucleotide are deoxyribonucleotides. In embodiments, all of the nucleotides in the polynucleotide are deoxyribonucleotides. In embodiments, none of the nucleotides in the polynucleotide are deoxyribonucleotides.

In embodiments, the polynucleotide comprises one or more 2′-modified nucleotides. In embodiments, the polynucleotide comprises 1, 2, 3, 4, 5, 6, 7, 8, 9, 10, 11, 12, 13, 14, 15, 16, 17, 18, 19, 20, 21, 22, 23, 24, 25, 50, 75, 100, or more 2′-modified nucleotides. In embodiments, the polynucleotide comprises 1-25, 25-50 or 1-50 2′-modified nucleotides. In embodiments, the polynucleotide comprises 15 2′-modified nucleotides. In embodiments, the polynucleotide comprises 20 2′-modified nucleotides. In embodiments, the polynucleotide comprises 25 2′-modified nucleotides. In embodiments, the polynucleotide comprises 30 2′-modified nucleotides. In embodiments, the polynucleotide comprises 35 2′-modified nucleotides. In embodiments, the polynucleotide comprises 40 2′-modified nucleotides. In embodiments, the polynucleotide comprises 45 2′-modified nucleotides. In embodiments, the polynucleotide comprises 50 2′-modified nucleotides. In embodiments, the polynucleotide comprises 60 2′-modified nucleotides. In embodiments, the polynucleotide comprises 70 2′-modified nucleotides. In embodiments, the polynucleotide comprises 80 2′-modified nucleotides. In embodiments, the polynucleotide comprises 90 2′-modified nucleotides. In embodiments, the polynucleotide comprises 1-100 2′-modified nucleotides. In embodiments, the polynucleotide comprises 10-75 2′-modified nucleotides. In embodiments, the polynucleotide comprises 25-50 2′-modified nucleotides.

In embodiments, about 10% or more of the nucleotides in the polynucleotide are 2′-modified nucleotides. In embodiments, about 20% or more of the nucleotides in the polynucleotide are 2′-modified nucleotides. In embodiments, about 30% or more of the nucleotides in the polynucleotide are 2′-modified nucleotides. In embodiments, about 40% or more of the nucleotides in the polynucleotide are 2′-modified nucleotides. In embodiments, about 50% or more of the nucleotides in the polynucleotide are 2′-modified nucleotides. In embodiments, about 60% or more of the nucleotides in the polynucleotide are 2′-modified nucleotides. In embodiments, about 70% or more of the nucleotides in the polynucleotide are 2′-modified nucleotides. In embodiments, about 80% or more of the nucleotides in the polynucleotide are 2′-modified nucleotides. In embodiments, about 90% or more of the nucleotides in the polynucleotide are 2′-modified nucleotides. In embodiments, all of the nucleotides in the polynucleotide are 2′-modified nucleotides. In embodiments, none of the nucleotides in the polynucleotide are 2′-modified nucleotides.

In embodiments, the one or more 2′-modified nucleotides are 2′-amine modified nucleotides. In embodiments, the one or more 2′-modified nucleotides are 2′-O-methyl modified nucleotides. In embodiments, the one or more 2′-modified nucleotides include a combination of 2′-amine modified nucleotides and 2′-O-methyl modified nucleotides.

In embodiments, the cyanine moiety is attached at the 5′-end of the polynucleotide.

In embodiments, the cyanine moiety is a streptocyanine moiety, a hemicyanine moiety, or a closed cyanine moiety.

In embodiments, the cyanine moiety has the formula:

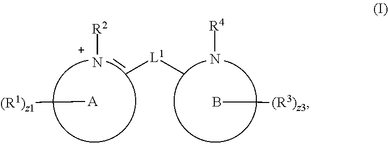

wherein R¹, R², R³, and R⁴ are independently hydrogen or substituted or unsubstituted alkyl. R⁴ and L¹ may optionally be joined to form a substituted or unsubstituted cycloalkyl, substituted or unsubstituted heterocycloalkyl. R² and L¹ may optionally be joined to form a substituted or unsubstituted cycloalkyl, substituted or unsubstituted heterocycloalkyl. L¹ is a covalent linker. Ring A and Ring B are independently heteroaryl. z1 and z3 are each independently an integer from 0 to 12. A person having ordinary skill in the art will recognize that the cyanine moiety may be in monovalent form when referred to as a portion of a the compound. The point of attachment to the remainder of the compound may be at R⁴ or R³.

In embodiments, the point of attachment of the cyanine moiety to the remainder of the compound is at R⁴. Thus, in embodiments, where R⁴ is a substituted alkyl, R⁴ is substituted with -L²-R⁵ wherein L² is a bond or covalent linker and R⁵ is a nucleic acid. Where R⁴ is a substituted alkyl and R⁴ is substituted with -L²-R⁵, a person having ordinary skill in the art will understand the formula refers to the compound. In embodiments, the nucleic acid is the polynucleotide comprising a nucleotide sequence that is fully complementary to a nucleotide sequence of a mitochondrial polynucleotide. Thus, in embodiments, the compound comprising a polynucleotide covalently linked to a cyanine moiety has the formula:

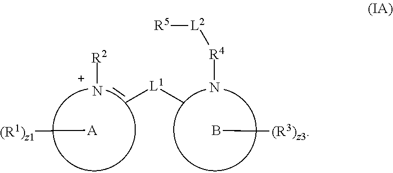

In Formula (IA), R¹, R², R³, R⁴, R⁵, L¹, L², z1 and z2 are as defined herein, including embodiments thereof. In embodiments, L² is attached to the 5′ oxygen of the nucleic acid (e.g. the polynucleotide comprising a nucleotide sequence that is fully complementary to a nucleotide sequence of a mitochondrial polynucleotide). In embodiments, L² is attached to the 3′ oxygen of the nucleic acid (e.g. the polynucleotide comprising a nucleotide sequence that is fully complementary to a nucleotide sequence of a mitochondrial polynucleotide). In embodiments, there is only one point of attachment between the cyanine moiety and the nucleic acid. In embodiments, the cyanine moiety is attached to a single nucleic acid. In embodiments, a nucleic acid is attached to multiple cyanine moieties (e.g., 2, 3, 4, 5, 10, or more cyanine moieties).

In embodiments, the point of attachment of the cyanine moiety to the remainder of the compound is at R³. Thus, in embodiments, where R³ is a substituted alkyl, R³ is substituted with -L²-R⁵ wherein L² is a bond or covalent linker and R⁵ is a nucleic acid. Where R³ is a substituted alkyl and R³ is substituted with -L²-R⁵, a person having ordinary skill in the art will understand the formula refers to the compound. In embodiments, the nucleic acid is the polynucleotide comprising a nucleotide sequence that is fully complementary to a nucleotide sequence of a mitochondrial polynucleotide. Thus, in embodiments, the compound comprising a polynucleotide covalently linked to a cyanine moiety has the formula:

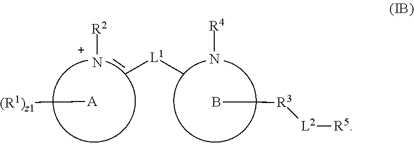

In Formula (IB) R¹, R², R³, R⁴, R⁵, L¹, L², z1 and z2 are as defined herein, including embodiments thereof. In embodiments, L² is attached to the 5′ oxygen of the nucleic acid (e.g. the polynucleotide comprising a nucleotide sequence that is fully complementary to a nucleotide sequence of a mitochondrial polynucleotide). In embodiments, L² is attached to the 3′ oxygen of the nucleic acid (e.g. the polynucleotide comprising a nucleotide sequence that is fully complementary to a nucleotide sequence of a mitochondrial polynucleotide).

In embodiments, L² is a bond, -L^{2A}-L^{2B}-L^{2C}-, —O—, —NH—, —S—, —C(O)—, —C(O)O—, —C(O)NH₂—, —OP(O)₂—, —OP(O)₂O—, —OP(S)(O)—, —OP(S)(O)O—, —OP(S)₂—, —OP(S)₂O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{2A}, L^{2B}, and L^{2C} are independently a bond, —O—, —NH—, —S—, —C(O)—, —C(O)O—, —C(O)NH₂—, —OP(O)₂—, —OP(O)₂O—, —OP(S)(O)—, —OP(S)(O)O—, —OP(S)₂—, —OP(S)₂O—, —S(O)—, —S(O)₂—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered). In embodiments, L^{2A}, L^{2B}, and L^{2C} are not all a bond. In embodiments, L^{2C} is attached to the nucleic acid portion of the compound (i.e. R⁵) and L^{2A} is attached to the cyanine moiety. In embodiments, where L² is substituted, L² is substituted with a substituent group. In embodiments, where L² is substituted, L² is substituted with a size-limited substituent group. In embodiments, where L² is substituted, L² is substituted with a lower substituent group. In embodiments, L² is a substituted (e.g. substituted with a substituent group, a size-limited substituent group or a lower substituent group) alkylene. In embodiments, L² is a substituted (e.g. substituted with a substituent group, a size-limited substituent group or a lower substituent group) heteroalkylene. In embodiments, L² is a substituted (e.g. substituted with a substituent group, a size-limited substituent group or a lower substituent group) cycloalkylene. In embodiments, L² is a substituted (e.g. substituted with a substituent group, a size-limited substituent group or a lower substituent group) heterocycloalkylene. In embodiments, L² is a substituted (e.g. substituted with a substituent group, a size-limited substituent group or a lower substituent group) arylene. In embodiments, L² is a substituted (e.g. substituted with a substituent group, a size-limited substituent group or a lower substituent group) heteroarylene. In embodiments, where L² is an alkylene, L² is a C₁-C₁₀ alkylene. In embodiments, where L² is a heteroalkylene, L² is a 2 to 10 membered heteroalkylene. In embodiments, where L² is a cycloalkylene, L² is a C₃-C₈ cycloalkylene. In embodiments, where L² is a heterocycloalkylene, L² is a 3 to 8 membered heterocycloalkylene. In embodiments, where L² is arylene, L² is a C₆ or C₁₀ arylene. In embodiments, where L² is a heteroarylene, L² is a 5, 6, 9 or 10 membered heteroarylene.

In embodiments, where L^{2A} is substituted, L^{2A} is substituted with a substituent group. In embodiments, where L^{2A} is substituted, L^{2A} is substituted with a size-limited substituent group. In embodiments, where L^{2A} is substituted, L^{2A} is substituted with a lower substituent group. In embodiments, L^{2A} is a substituted (e.g. substituted with a substituent group, a size-limited substituent group or a lower substituent group) alkylene. In embodiments, L^{2A} is a substituted (e.g. substituted with a substituent group, a size-limited substituent group or a lower substituent group) heteroalkylene. In embodiments, L^{2A} is a substituted (e.g. substituted with a substituent group, a size-limited substituent group or a lower substituent group) cycloalkylene. In embodiments, L^{2A} is a substituted (e.g. substituted with a substituent group, a size-limited substituent group or a lower substituent group) heterocycloalkylene. In embodiments, L^{2A} is a substituted (e.g. substituted with a substituent group, a size-limited substituent group or a lower substituent group) arylene. In embodiments, L^{2A} is a substituted (e.g. substituted with a substituent group, a size-limited substituent group or a lower substituent group) heteroarylene. In embodiments, where L^{2A} is an alkylene, L^{2A} is a C₁-C₁₀ alkylene. In embodiments, where L^{2A} is a heteroalkylene, L^{2A} is a 2 to 10 membered heteroalkylene. In embodiments, where L^{2A} is a cycloalkylene, L^{2A} is a C₃-C₈ cycloalkylene. In embodiments, where L^{2A} is a heterocycloalkylene, L^{2A} is a 3 to 8 membered heterocycloalkylene. In embodiments, where L^{2A} is arylene, L^{2A} is a C₆ or C₁₀ arylene. In embodiments, where L^{2A} is a heteroarylene, L^{2A} is a 5, 6, 9 or 10 membered heteroarylene.

In embodiments, L^{2B} is a bond, —O—, —NH—, —S—, —C(O)—, —C(O)O—, —C(O)NH₂—, —OP(O)₂—, —OP(O)₂₀—, —OP(S)(O)—, —OP(S)(O)O—, —OP(S)₂—, —OP(S)₂O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, where L^{2B} is substituted, L^{2B} is substituted with a substituent group. In embodiments, where L^{2B} is substituted, L^{2B} is substituted with a size-limited substituent group. In embodiments, where L^{2B} is substituted, L^{2B} is substituted with a lower substituent group. In embodiments, L^{2B} is a substituted (e.g. substituted with a substituent group, a size-limited substituent group or a lower substituent group) alkylene. In embodiments, L^{2B} is a substituted (e.g. substituted with a substituent group, a size-limited substituent group or a lower substituent group) heteroalkylene. In embodiments, L^{2B} is a substituted (e.g. substituted with a substituent group, a size-limited substituent group or a lower substituent group) cycloalkylene. In embodiments, L^{2B} is a substituted (e.g. substituted with a substituent group, a size-limited substituent group or a lower substituent group) heterocycloalkylene. In embodiments, L^{2B} is a substituted (e.g. substituted with a substituent group, a size-limited substituent group or a lower substituent group) arylene. In embodiments, L^{2B} is a substituted (e.g. substituted with a substituent group, a size-limited substituent group or a lower substituent group) heteroarylene. In embodiments, where L^{2B} is an alkylene, L^{2B} is a C₁-C₁₀ alkylene. In embodiments, where L^{2B} is a heteroalkylene, L^{2B} is a 2 to 10 membered heteroalkylene. In embodiments, where L^{2B} is a cycloalkylene, L^{2B} is a C₃-C₈ cycloalkylene. In embodiments, where L^{2B} is a heterocycloalkylene, L^{2B} is a 3 to 8 membered heterocycloalkylene. In embodiments, where L^{2B} is arylene, L^{2B} is a C₆ or C₁₀ arylene. In embodiments, where L^{2B} is a heteroarylene, L² is a 5, 6, 9 or 10 membered heteroarylene.

In embodiments, L^{2C} is a bond, —O—, —NH—, —S—, —C(O)—, —C(O)O—, —C(O)NH₂—, —OP(O)₂—, —OP(O)₂O—, —OP(S)(O)—, —OP(S)(O)O—, —OP(S)₂—, —OP(S)₂O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, where L^{2C} is substituted, L^{2C} is substituted with a substituent group. In embodiments, where L^{2C} is substituted, L^{2C} is substituted with a size-limited substituent group. In embodiments, where L^{2C} is substituted, L^{2C} is substituted with a lower substituent group. In embodiments, L^{2C} is a substituted (e.g. substituted with a substituent group, a size-limited substituent group or a lower substituent group) alkylene. In embodiments, L^{2C} is a substituted (e.g. substituted with a substituent group, a size-limited substituent group or a lower substituent group) heteroalkylene. In embodiments, L^{2C} is a substituted (e.g. substituted with a substituent group, a size-limited substituent group or a lower substituent group) cycloalkylene. In embodiments, L^{2C} is a substituted (e.g. substituted with a substituent group, a size-limited substituent group or a lower substituent group) heterocycloalkylene. In embodiments, L^{2C} is a substituted (e.g. substituted with a substituent group, a size-limited substituent group or a lower substituent group) arylene. In embodiments, L^{2C} is a substituted (e.g. substituted with a substituent group, a size-limited substituent group or a lower substituent group) heteroarylene. In embodiments, L^{2C} is —OP(O)₂—, wherein the phosphorus atom is attached to the 5′ oxygen of the nucleic acid (e.g. the polynucleotide comprising a nucleotide sequence that is fully complementary to a nucleotide sequence of a mitochondrial polynucleotide). In embodiments, L^{2C} is —OP(O)₂—, wherein the phosphorus atom is attached to the 3′ oxygen of the nucleic acid (e.g. the polynucleotide comprising a nucleotide sequence that is fully complementary to a nucleotide sequence of a mitochondrial polynucleotide). In embodiments, where L^{2C} is an alkylene, L^{2C} is a C₁-C₁₀ alkylene. In embodiments, where L^{2C} is a heteroalkylene, L^{2C} is a 2 to 10 membered heteroalkylene. In embodiments, where L^{2C} is a cycloalkylene, L² is a C₃-C₈ cycloalkylene. In embodiments, where L^{2C} is a heterocycloalkylene, L^{2C} is a 3 to 8 membered heterocycloalkylene. In embodiments, where L^{2C} is arylene, L^{2C} is a C₆ or C₁₀ arylene. In embodiments, where L^{2C} is a heteroarylene, L^{2C} is a 5, 6, 9 or 10 membered heteroarylene.

In embodiments, L¹ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is a substituted or unsubstituted alkenylene. In embodiments, L¹ is a substituted or unsubstituted cycloalkenylene.

In embodiments, L¹ is a -L^{1A}-L^{1B}-L^{1C}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, where L¹ is substituted, L¹ is substituted with a substituent group. In embodiments, where L¹ is substituted, L¹ is substituted with a size-limited substituent group. In embodiments, where L¹ is substituted, L¹ is substituted with a lower substituent group. In embodiments, L¹ is a substituted (e.g. substituted with a substituent group, a size-limited substituent group or a lower substituent group) alkylene. In embodiments, L¹ is a substituted (e.g. substituted with a substituent group, a size-limited substituent group or a lower substituent group) heteroalkylene. In embodiments, L¹ is a substituted (e.g. substituted with a substituent group, a size-limited substituent group or a lower substituent group) cycloalkylene. In embodiments, L¹ is a substituted (e.g. substituted with a substituent group, a size-limited substituent group or a lower substituent group) heterocycloalkylene. In embodiments, L¹ is a substituted (e.g. substituted with a substituent group, a size-limited substituent group or a lower substituent group) arylene. In embodiments, L¹ is a substituted (e.g. substituted with a substituent group, a size-limited substituent group or a lower substituent group) heteroarylene.

In embodiments, L¹ has the formula -L^{1A}-L^{1B}-L^{1C}-, wherein L^{1A}, L^{1B}, and L^{1C} are independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{1A} is a substituted or unsubstituted alkenylene. In embodiments, L^{1A} is a substituted or unsubstituted cycloalkenylene. In embodiments, L^{1B} is a substituted or unsubstituted alkenylene. In embodiments, L^{1B} is a substituted or unsubstituted cycloalkenylene. In embodiments, L^{1C} is a substituted or unsubstituted alkenylene. In embodiments, L^{1C} is a substituted or unsubstituted cycloalkenylene.

In embodiments, L¹, L^{1A}, L^{1B}, and L^{1C} are each independently substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹, L^{1A}, L^{1B}, and L^{1c} are each independently substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹, L^{1A}, L^{1B}, and L^{1C} are each independently unsubstituted alkylene, unsubstituted heteroalkylene, unsubstituted cycloalkylene, unsubstituted heterocycloalkylene, unsubstituted arylene, or unsubstituted heteroarylene.

In embodiments, L¹, L^{1A}, L^{1B}, and L^{1C} are each independently unsubstituted alkenylene, unsubstituted cycloalkenylene, or unsubstituted heterocycloalkenylene.

In embodiments, the cyanine moiety has the formula:

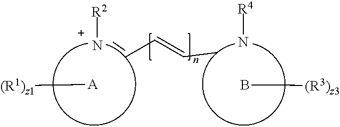

wherein n is an integer from 1 to 20. In embodiments, R¹ is hydrogen, methyl, ethyl, propyl, or butyl. In embodiments, R² is hydrogen, methyl, ethyl, propyl, or butyl. In embodiments, R³ is hydrogen, methyl, ethyl, propyl, or butyl. In embodiments, R⁴ is hydrogen, methyl, ethyl, propyl, or butyl. This formula may be alternatively drawn as:

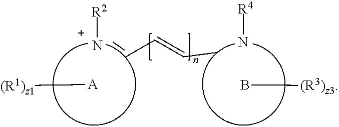

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

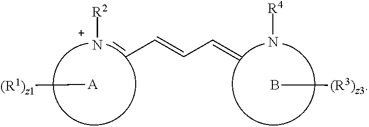

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

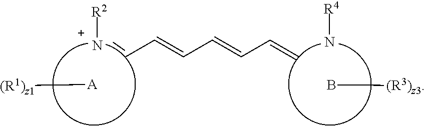

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

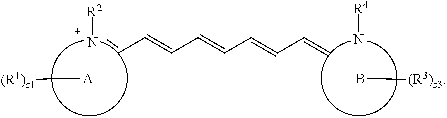

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

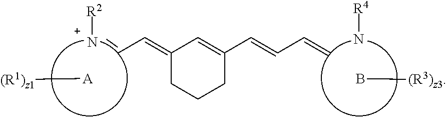

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

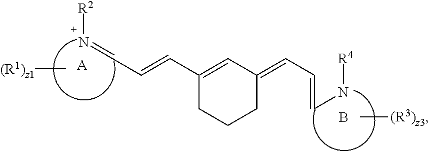

which may be alternatively drawn as:

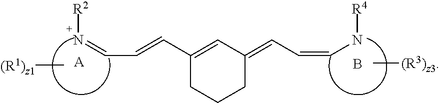

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

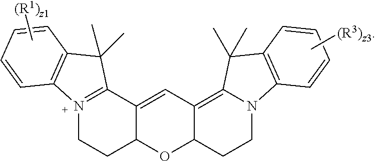

R¹, R², R³, R⁴, R⁵, L¹, L², z1 and z2 are as defined herein, including embodiments thereof. In embodiments, where R³ is a substituted alkyl, R³ is substituted with -L²-R⁵. In embodiments, z1 is 0.

In embodiments, the cyanine moiety has the formula:

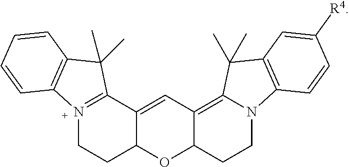

R¹, R², R³, R⁴, R⁵, L¹, L², z1 and z2 are as defined herein, including embodiments thereof. In embodiments, where R⁴ is a substituted alkyl, R⁴ is substituted with -L²-R⁵.

In embodiments, the cyanine moiety has the formula:

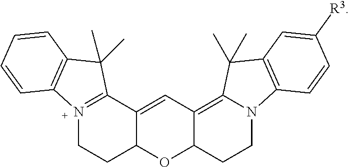

R¹, R², R³, R⁴, R⁵, L¹, L², z1 and z2 are as defined herein, including embodiments thereof. In embodiments, where R³ is a substituted alkyl, R³ is substituted with -L²-R⁵.

In embodiments, Ring A is pyrrolyl, imidazolyl, thiazolyl, pyridinyl, quinolinyl, indolyl, or benzothiazolyl. In embodiments, Ring B is pyrrolyl, imidazolyl, thiazolyl, pyridinyl, quinolinyl, indolyl, or benzothiazolyl.

In embodiments, Ring A is

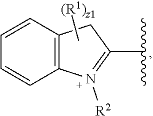

wherein R¹, R², and z1 are as described herein. In embodiments, Ring A is

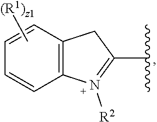

wherein R¹, R², and z1 are as described herein. In embodiments, Ring A is

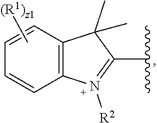

wherein R¹, R², and z1 are as described herein. In embodiments, Ring A is

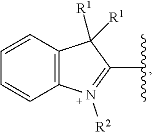

wherein R¹ and R² are as described herein. In embodiments, Ring A is

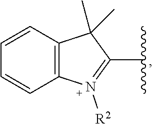

wherein R² is as described herein. In embodiments, Ring A is

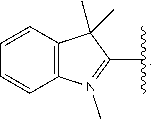

In embodiments, Ring A is

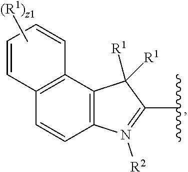

wherein R¹ and R² are as described herein. In embodiments, Ring A is

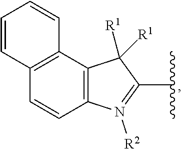

wherein R¹ and R² are as described herein. In embodiments, Ring A is

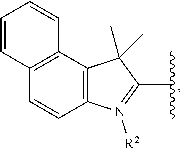

wherein R² is as described herein. In embodiments, Ring A is

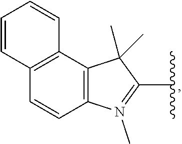

wherein R¹ and R² are as described herein.

In embodiments, Ring B is

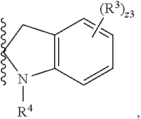

wherein R³, R⁴, and z3 are as described herein. In embodiments, Ring B is

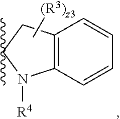

wherein R³, R⁴, and z3 are as described herein. In embodiments, z3 is 0. In embodiments, Ring B is

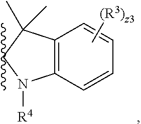

wherein R³, R⁴, and z3 are as described herein. In embodiments, z3 is 0. In embodiments, Ring B is

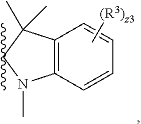

wherein R³ and z3 are as described herein. In embodiments, Ring B is

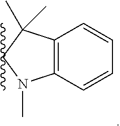

In embodiments, Ring B is

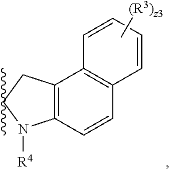

wherein R³, R⁴, and z3 are as described herein. In embodiments, z3 is 0. In embodiments, Ring B is

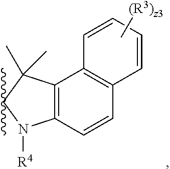

wherein R³, R⁴, and z3 are as described herein. In embodiments, z3 is 0. In embodiments, Ring B is

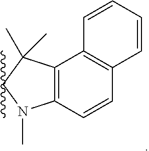

In embodiments, Ring B is

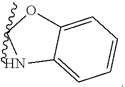

In embodiments, z3 is 0. In embodiments, Ring B is

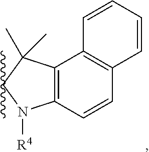

wherein R⁴ is as described herein. In embodiments, Ring B is

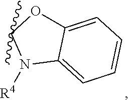

wherein R⁴ is as described herein. In embodiments, Ring B is

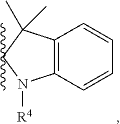

wherein R⁴ is as described herein.

In embodiments, R⁴ and L¹ may optionally be joined to form a substituted or unsubstituted C₆ cycloalkyl, substituted or unsubstituted 6 membered heterocycloalkyl. R² and L¹ may optionally be joined to form a substituted or unsubstituted C₆ cycloalkyl, substituted or unsubstituted 6 membered heterocycloalkyl. In embodiments, where R⁴ and L¹ are joined to form a substituted cycloalkyl, the substituted cycloalkyl is substituted with a substituent group. In embodiments, where R⁴ and L¹ are joined to form a substituted cycloalkyl, the substituted cycloalkyl is substituted with a size-limited substituent group. In embodiments, where R⁴ and L¹ are joined to form a substituted cycloalkyl, the substituted cycloalkyl is substituted with a lower substituent group.

In embodiments, where R⁴ and L¹ are joined to form a substituted heterocycloalkyl, the substituted heterocycloalkyl is substituted with a substituent group. In embodiments, where R⁴ and L¹ are joined to form a substituted heterocycloalkyl, the substituted heterocycloalkyl is substituted with a size-limited substituent group. In embodiments, where R⁴ and L¹ are joined to form a substituted heterocycloalkyl, the substituted hetero cycloalkyl is substituted with a lower substituent group.

In embodiments, the cyanine moiety comprises one of the following structures:

(i) R₁R₂N⁺═CH[CH═CH]ₙ—NR₁R₂ (Formula I);

(ii) Aryl=N⁺═CH[CH═CH]ₙ—NR₁R₂ (Formula II); or

(iii) Aryl=N⁺═CH[CH═CH]ₙ—N=Aryl (Formula III),
wherein
   n is an integer from 1-20;
   R₁ is hydrogen, methyl, ethyl, propyl, or butyl; and
   R² is hydrogen, methyl, ethyl, propyl, or butyl.

In embodiments, N=Aryl or Aryl=N⁺ is an heteroaromatic moiety comprising the N or N⁺, wherein the heteroaromatic moiety is pyrrole, imidazole, thiazole, pyridine, quinoline, indole, or benzothiazole.

In embodiments, the cyanine moiety is fluorescent. In embodiments, the cyanine moiety is not fluorescent.

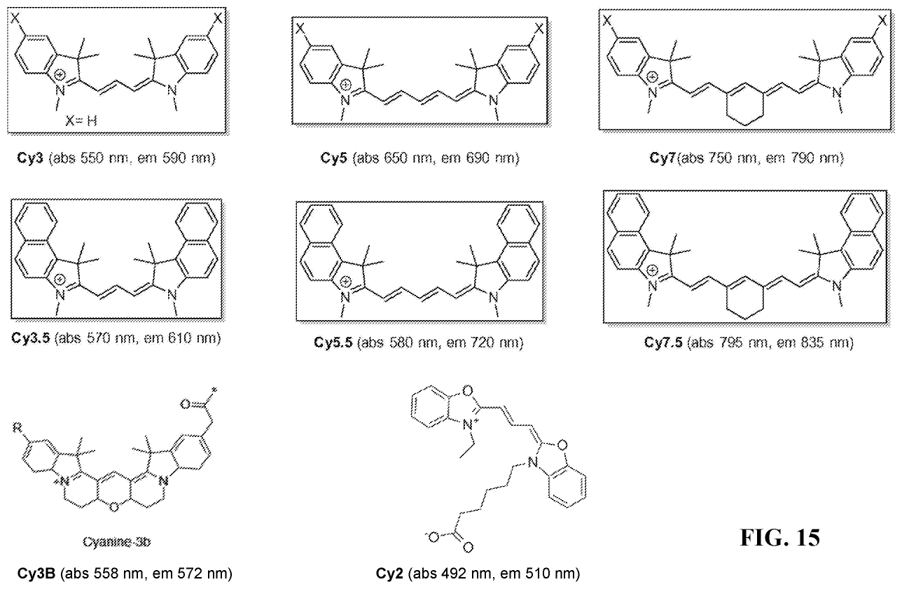

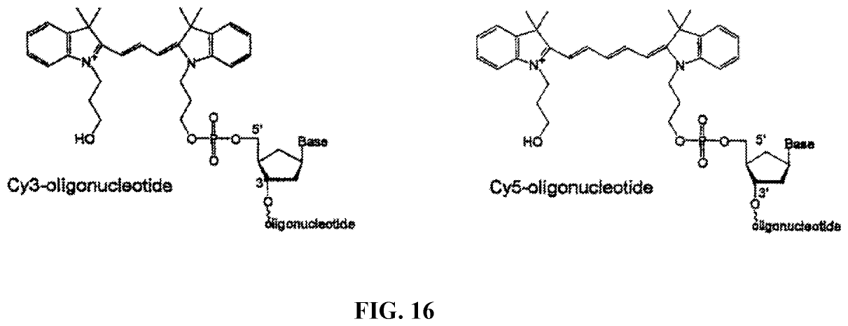

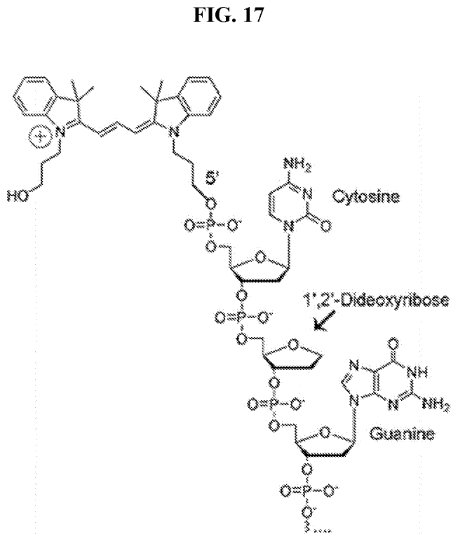

In embodiments, the cyanine moiety is a Cy2 moiety, Cy3 moiety, Cy3B moiety, Cy3.5 moiety, Cy5 moiety, Cy5.5 moiety, Cy7.5 moiety, or Cy7 moiety. In embodiments, the cyanine moiety comprises a structure selected from a structure shown in FIG. 15. Structures designated in FIG. 15 as Cy3, Cy5, Cy7, Cy3.5, Cy5.5, Cy7.5, Cy3B, and Cy2 are also referred to herein as Formulas IV-XI, respectively. In embodiments, one or both “X” in Formulas IV-VI are hydrogen. In embodiments, “R” in Formula X is hydrogen. Non-limiting examples of covalent linkages between a cyanine moiety and an oligonucleotide are illustrated in FIG. 16. The exemplary nucleotides in FIG. 16 are DNA nucleotides. Covalent linkage to other nucleotide bases, including RNA or modified nucleotides, are also contemplated herein. FIG. 17 illustrates a further non-limiting example of a covalent linkage between a cyanine moiety and an oligonucleotide.

In embodiments, the compound is

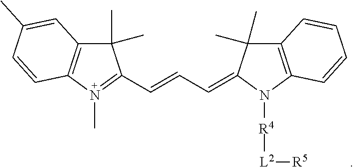

In embodiments, the compound is

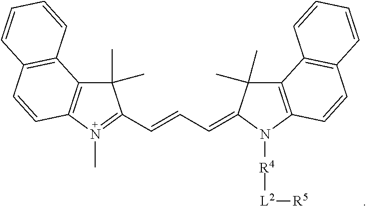

In embodiments, the compound is

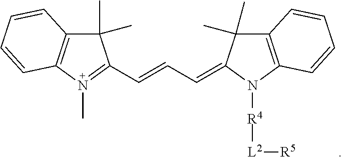

In embodiments, the compound is

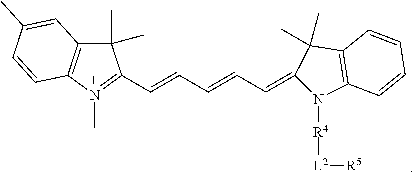

In embodiments, the compound is

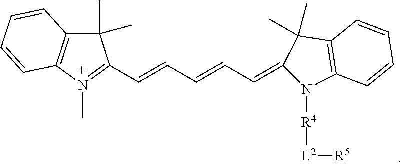

In embodiments, the compound is

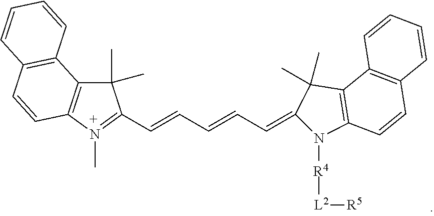

In embodiments, the compound is

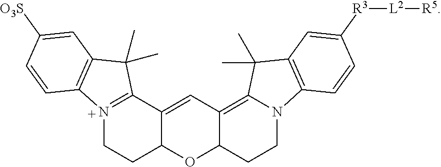

In embodiments, the compound is

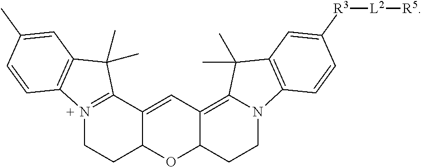

In embodiments, the compound is

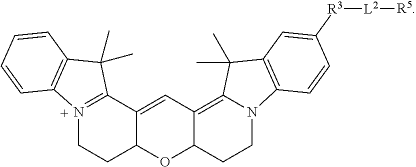

In embodiments, the compound is

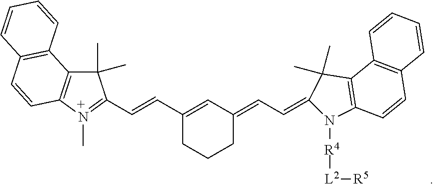

In embodiments, the compound is

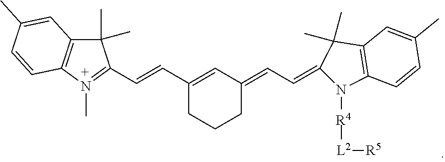

In embodiments, the compound is

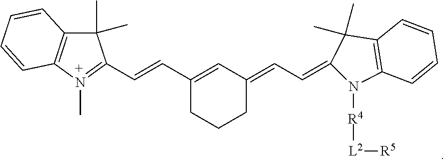

In embodiments, the compound is

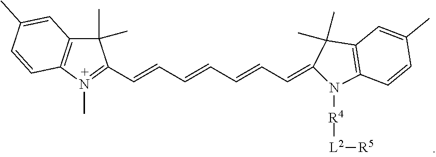

In embodiments, the compound is

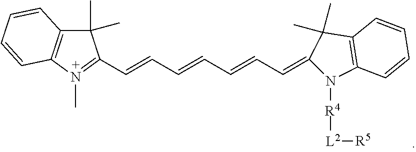

In embodiments, the compound is

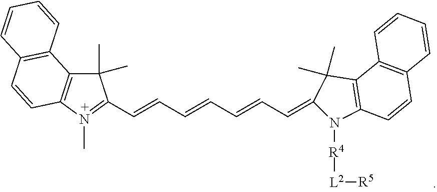

In embodiments, the compound is

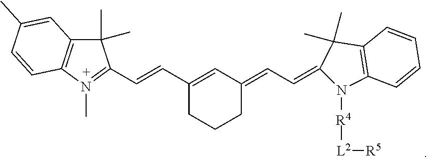

In embodiments, the compound is

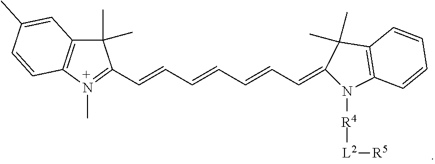

In embodiments, the compound is

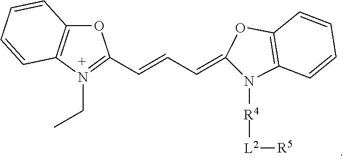

In embodiments, at least about 5%, 10%, 15%, 20%, 25%, 30%, 40%, 50%, 60%, 70%, 80%, or 90% of the nucleotides in the polynucleotide are deoxyribonucleotides. In embodiments, the polynucleotide is a polyribonucleotide (e.g., 100% of the nucleotides in the polynucleotide are deoxyribonucleotides). In embodiments, the polynucleotide is a polydeoxyribonucleotide (e.g., 100% of the nucleotides in the polynucleotide are deoxyribonucleotides). In embodiments, at least about 5%, 10%, 15%, 20%, 25%, 30%, 40%, 50%, 60%, 70%, 80%, or 90% of the nucleotides in the polynucleotide are ribonucleotides. In embodiments, the polynucleotide is a polyribonucleotide (e.g., 100% of the nucleotides in the polynucleotide are ribonucleotides). In embodiments, the polynucleotide comprises a combination of ribonucleotides and deoxyribonucleotides. In embodiments, the polynucleotide further comprises modified nucleotides, such as 2′-modified nucleotides. In embodiments, the polynucleotide (e.g., a polydeoxyribonucleotide, a polyribonucleotide, or a polynucleotide comprising a mixture of deoxyribonucleotides, ribonucleotides, and/or 2′-modified nucleotides), has a linkage other than a phosphodiester bond between at least one pair of linked nucleotides. In embodiments, all of the nucleotides in the polynucleotide are linked by a phosphodiester bond. In embodiments, at least one pair of linked nucleotides in the polynucleotide are linked by a phosphodiester bond. In embodiments, 1, 2, 3, 4, 5, 6, 7, 8, 9, 10, 10-15, 15-20, 20-25, or 25-30 pairs of linked nucleotides are linked by a bond other than a phosphodiester bond. In embodiments, the polynucleotide comprises one or more phosphorothioate, methylphosphonate, and -anomeric sugar-phosphate nucleotides (e. g., modified deoxynucleotides, modified ribonucleotides, and/or further modified 2′-modified nucleotides). In embodiments, the polynucleotide does not comprise a phosphorothioate linker between any nucleotides. In embodiments, less than all of the polynucleotides are connected by a phosphorothioate linker. In embodiments, less than 100%, 90%, 80%, 70%, 60%, 50%, 40%, 30%, 20%, 10%, or 5% of the linkages between nucleotides are phosphorothioate linkages. In embodiments, the polynucleotide does not comprise 2′-fluoro modified nucleotides. In embodiments, the polynucleotide comprises at least one nucleotide that is not a 2′-fluoro modified nucleotide. In embodiments, if the polynucleotide comprises cytosines and/or uracil, then at least one of the cytosines and/or uracils is not a 2′-fluoro modified nucleotide. In embodiments, if the polynucleotide comprises cytosines and/or uracils, then less than 100%, 90%, 80%, 70%, 60%, 50%, 40%, 30%, 20%, 10%, or 5% of the cytosines and/or uracils are not a 2′-fluoro modified nucleotide. In embodiments, the polynucleotide comprises 2′-fluoro (2′F), 2′-O-methyl (OMe), 2′-O-ethyl (cET), phosphorothioate linkages (PS), and/or locked nucleic acid (LNA) modifications.

In embodiments, the polynucleotide is single stranded. In embodiments, the polynucleotide is double stranded.

In embodiments, the polynucleotide is about 10-200 nucleotides in length. In embodiments, the polynucleotide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length. In embodiments, the polynucleotide is 10-25, 11-25, 12-25, 13-25, 14-25, 15-25, 15-30, 15-35, 15-40, 15-45, 15-50, 20-30, 25-50, 25-75, 50-75, 50-100, 75-100, or 100-150 nucleotides in length. In embodiments, the polynucleotide is less than 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In embodiments, the polynucleotide is about 10 nucleotides in length or more. In embodiments, the polynucleotide is about 15 nucleotides in length or more. In embodiments, the polynucleotide is about 20 nucleotides in length or more. In embodiments, the polynucleotide is about 25 nucleotides in length or more. In embodiments, the polynucleotide is about 30 nucleotides in length or more. In embodiments, the polynucleotide is about 35 nucleotides in length or more. In embodiments, the polynucleotide is about 40 nucleotides in length or more. In embodiments, the polynucleotide is about 45 nucleotides in length or more. In embodiments, the polynucleotide is about 50 nucleotides in length or more. In embodiments, the polynucleotide is about 55 nucleotides in length or more. In embodiments, the polynucleotide is about 60 nucleotides in length or more. In embodiments, the polynucleotide is about 65 nucleotides in length or more. In embodiments, the polynucleotide is about 70 nucleotides in length or more. In embodiments, the polynucleotide is about 75 nucleotides in length or more. In embodiments, the polynucleotide is about 80 nucleotides in length or more. In embodiments, the polynucleotide is about 85 nucleotides in length or more. In embodiments, the polynucleotide is about 90 nucleotides in length or more. In embodiments, the polynucleotide is about 95 nucleotides in length or more. In embodiments, the polynucleotide is about 100 nucleotides in length or more.

In embodiments, the polynucleotide is about 10 nucleotides in length or less. In embodiments, the polynucleotide is about 15 nucleotides in length or less. In embodiments, the polynucleotide is about 20 nucleotides in length or less. In embodiments, the polynucleotide is about 25 nucleotides in length or less. In embodiments, the polynucleotide is about 30 nucleotides in length or less. In embodiments, the polynucleotide is about 35 nucleotides in length or less. In embodiments, the polynucleotide is about 40 nucleotides in length or less. In embodiments, the polynucleotide is about 45 nucleotides in length or less. In embodiments, the polynucleotide is about 50 nucleotides in length or less. In embodiments, the polynucleotide is about 55 nucleotides in length or less. In embodiments, the polynucleotide is about 60 nucleotides in length or less. In embodiments, the polynucleotide is about 65 nucleotides in length or less. In embodiments, the polynucleotide is about 70 nucleotides in length or less. In embodiments, the polynucleotide is about 75 nucleotides in length or less. In embodiments, the polynucleotide is about 80 nucleotides in length or less. In embodiments, the polynucleotide is about 85 nucleotides in length or less. In embodiments, the polynucleotide is about 90 nucleotides in length or less. In embodiments, the polynucleotide is about 95 nucleotides in length or less. In embodiments, the polynucleotide is about 100 nucleotides in length or less.

In embodiments, the polynucleotide is 1-200 nucleotides in length. In embodiments, the polynucleotide is 10-150 nucleotides in length. In embodiments, the polynucleotide is 15-125 nucleotides in length. In embodiments, the polynucleotide is 20-100 nucleotides in length. In embodiments, the polynucleotide is 25-75 nucleotides in length. In embodiments, the polynucleotide is 1-100 nucleotides in length. In embodiments, the polynucleotide is 10-75 nucleotides in length. In embodiments, the polynucleotide is 15-50 nucleotides in length.

In embodiments, the polynucleotide further comprises another cyanine moiety attached at the 3′-end of the polynucleotide (e.g., to the oxygen at the 3′ end of the polynucleotide). In embodiments, the cyanine moiety that is attached to the 5′-end of the polynucleotide is different than the cyanine moiety that is attached to the 3′-end of the polynucleotide. In embodiments, the cyanine moiety that is attached to the 5′-end of the polynucleotide is the same as the cyanine moiety that is attached to the 3′-end of the polynucleotide. In embodiments, there is no cyanine moiety at the 3′-end of the polynucleotide.

In embodiments, the polynucleotide is covalently linked to one or more cyanine moieties through a bioconjugate linker (e.g., as a result of a reaction between two bioconjugate reactive moieties). In embodiments, the polynucleotide is covalently linked to one or more cyanine moieties via a N-hydroxysuccinimide (NHS) ester linkage, a sulfo-NHS linkage, a hydroxybenzotriazole (HOBt) linkage, a 1-hydroxy-7-azabenzotriazole (HOAt) linkage, or a pentafluorophenol linkage. In embodiments, the polynucleotide is covalently linked to one or more cyanine moieties via a phosphoramidite linkage. In embodiments, the covalent linkage comprises an ester bond, a disulfide bond, or a bond that has been formed as a result of a click reaction. Non-limiting examples of click reactions include reactions between an azide and an alkyne; an alkyne and a strained difluorooctyne; a diaryl-cyclooctyne and a 1,3-nitrone; a cyclooctene, trans-cycloalkene, or oxanorbornadiene and an azide, tetrazine, or tetrazole; an activated alkene or oxanorbornadiene and an azide; a strained cyclooctene or other activated alkene and a tetrazine; or a tetrazole that has been activated by ultraviolet light and an alkene.

As used herein, the term “bioconjugate reactive moiety” and “bioconjugate” refers to the resulting association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

In embodiments, the polynucleotide is a CRISPR/Cas9 guide RNA (e.g., an sgRNA, a crRNA, or a tracrRNA), an RNA interference polynucleotide, or an antisense oligonucleotide.

In an aspect, included herein is cell comprising a compound or complex disclosed herein.

In an aspect, included herein is a complex comprising a protein and a compound disclosed herein. In embodiments, the protein is an RNA-guided protein. In embodiments, the RNA-guided protein is an RNA-guided enzyme. In embodiments, the RNA-guided enzyme is an RNA-guided endonuclease enzyme.

In embodiments the RNA-guided protein comprises a mitochondrial localization amino acid sequence covalently attached to N-terminus thereof.

In embodiments, the RNA-guided endonuclease is a Type II or a Type V CRISPR effector endonuclease. In embodiments, the RNA-guided endonuclease enzyme is a Cas9, a Cpf1 (also known as Cas12a), or a variant thereof. Non-limiting examples of Type II CRISPR endonucleases include Streptococcus pyogenes Cas9 (SpCas9), Staphylococcus aureus Cas9 (SaCas9), and Neisseria meningitides Cas9 (NmCas9). Non-limiting examples of Type V CRISPR endonucleases include Lachnospiraceae bacterium (LbAsCpf1), and Acidaminococcus Cpf1 (AsCpf1).

In embodiments, the Cpf1 is from an Acidaminococcus sp. BV3L6 or Lachnospiraceae bacterium ND2006 (AsCpf1 and LbCpf1, respectively). Cpf1 is both a DNA and RNA endonuclease, and is commonly referred to as an RNA-guided endonuclease. In embodiments, the Cas9 is Strepyogenes Cas9 (Sp Cas9) or Staphylococcus aureus Cas9 (SaCas9).

In embodiments, a polynucleotide provided herein is used in a CRISPR system to activate, silence, reduce the expression of, or base-edit a mitochondrial gene or polynucleotide. For example, a CRISPR endonuclease can be fused to an effector protein, such as a transcriptional activating protein (e.g., RelA, or VP64), or a silencing protein (e.g., KRAB). In embodiments, a CRISPR endonuclease fused to an effector protein bears one or more mutations attenuating or eliminating DNA cleavage activity of the CRISPR endonuclease. In embodiments, the CRISPR endonuclease is fused to an activating domain. Examples of activating domains include, without limitation, TFAM, TFB1M, and TFB2M. In embodiments, the CRISPR endonuclease is fused to a silencing domain. Non-limiting examples of silencing domains include defective versions of TFAM, TFB1M, and TFB2M, bearing mutations that attenuate or eliminate a transcriptional activation ability, thereby competitively inhibiting non-defective versions thereof.

Various aspects of the CRISPR-Cas system are known in the art. Non-limiting aspects of this system are described, e.g., in U.S. Pat. No. 9,023,649, issued May 5, 2015; U.S. Pat. No. 9,074,199, issued Jul. 7, 2015; U.S. Pat. No. 8,697,359, issued Apr. 15, 2014; U.S. Pat. No. 8,932,814, issued Jan. 13, 2015; U.S. Application Publication No. 2016/0298096, published Oct. 13, 2016; Cho et al., (2013) Nature Biotechnology Vol 31 No 3 pp 230-232 (including supplementary information); and Jinek et al., (2012) Science Vol 337 No 6096 pp 816-821, the entire contents of each of which are incorporated herein by reference.

The Type II CRISPR is one of the most well characterized systems and carries out targeted double-stranded breaks in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In engineered CRISPR/Cas9 systems, single guide RNA (“gRNA”) may replace crRNA and tracrRNA with a single RNA construct that includes the protospacer element and a linker loop sequence. Use of gRNA may simplify the components needed to use CRISPR/Cas9 for genome editing. The Cas9 species of different organisms have different PAM sequences. For example, Streptococcus pyogenes (Sp) has a PAM sequence of 5′-NGG-3′ (SEQ ID NO:46), Staphylococcus aureus (Sa) has a PAM sequence of 5′-NGRRT-3′ (SEQ ID NO:47) or 5′-NGRRN-3′ (SEQ ID NO:48), Neisseria meningitidis (NM) has a PAM sequence of 5′-NNNNGATT-3′ (SEQ ID NO:49), Streptococcus thermophilus (St) has a PAM sequence of 5′-NNAGAAW-3′ (SEQ ID NO:50), Treponema denticola (Td) has a PAM sequence of 5′-NAAAAC-3′ (SEQ ID NO:51). Cas9 mediates cleavage of target DNA to create a DSB within the protospacer. Activity of the CRISPR/Cas system in nature comprises three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien polynucleotide. The alien polynucleotides come from viruses attaching the bacterial cell. Thus, in the bacterial cell, several of the so-called ‘Cm’ proteins are involved with the natural function of the CRISPR/Cas system and serve roles in functions such as insertion of the alien DNA, etc. CRISPR may also function with nucleases other than Cas9. Two genes from the Cpf1 family contain a RuvC-like endonuclease domain, but they lack Cas9's second HNH endonuclease domain. Cpf1 cleaves DNA in a staggered pattern and requires only one RNA rather than the two (tracrRNA and crRNA) needed by Cas9 for cleavage. Cpf1's preferred PAM is 5′-TTN (SEQ ID NO:52), differing from that of Cas9 (3′-NGG (SEQ ID NO:53)) in both genomic location and GC-content. Mature crRNAs for Cpf1-mediated cleavage are 42-44 nucleotides in length, about the same size as Cas9's, but with the direct repeat preceding the spacer rather than following it. The Cpf1 crRNA is also much simpler in structure than Cas9's; only a short stem-loop structure in the direct repeat region is necessary for cleavage of a target. Cpf1 also does not require an additional tracrRNA. Whereas Cas9 generates blunt ends 3 nt upstream of the PAM site, Cpf1 cleaves in a staggered fashion, creating a five nucleotide 5′ overhang 18-23 nt away from the PAM. Other CRISPR-associated proteins besides Cas9 may be used instead of Cas9. For example, CRISPR-associated protein 1 (Cm′) is one of the two universally conserved proteins found in the CRISPR prokaryotic immune defense system. Cas1 is a metal-dependent DNA-specific endonuclease that produces double-stranded DNA fragments. Cas1 forms a stable complex with the other universally conserved CRISPR-associated protein, Cas2, which is part of spacer acquisition for CRISPR systems.

There are also CRISPR/Cas9 variants that do not use a PAM sequence such as NgAgo. NgAgo functions with a 24-nucleotide ssDNA guide and is believed to cut 8-11 nucleotides from the start of this sequence. The ssDNA is loaded as the protein folds and cannot be swapped to a different guide unless the temperature is increased to non-physiological 55° C. A few nucleotides in the target DNA are removed near the cut site. Techniques for using NgAgo are described in Gao, F. et al., DNA-guided Genome Editing Using the Natronobacterium Gregoryi Argonaute, 34 Nature Biotechnology 768 (2016), the entire content of which is incorporated herein by reference. DSBs may be formed by making two single-stranded breaks at different locations creating a cut DNA molecule with sticky ends.

Single-strand breaks or “nicks” may be formed by modified versions of the Cas9 enzyme containing only one active catalytic domain (called “Cas9 nickase”). Cas9 nickases still bind DNA based on gRNA specificity, but nickases are only capable of cutting one of the DNA strands. Two nickases targeting opposite strands are required to generate a DSB within the target DNA (often referred to as a “double nick” or “dual nickase” CRISPR system). This requirement dramatically increases target specificity, since it is unlikely that two off-target nicks will be generated within close enough proximity to cause a DSB. Techniques for using a dual nickase CRISPR system to create a DSB are described in Ran, et al., Double Nicking by RNA-Guided CRISPR Cas9 for Enhanced Genome Editing Specificity, 154 Cell 6:1380 (2013), the entire content of which is incorporated herein by reference.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, as well as homologs and modified versions thereof. These enzymes are known; for example, the amino acid sequence of S. pyogenes Cas9 protein may be found in the SwissProt database under accession number Q99ZW2 (SEQ ID NO:19) and in the NCBI database as under accession number Q99ZW2.1. UniProt database accession numbers A0A0G4DEU5 and CDJ55032 (SEQ ID NO:54) provide another example of a Cas9 protein amino acid sequence. Another non-limiting example is a Streptococcus thermophilus Cas9 protein, the amino acid sequence of which may be found in the UniProt database under accession number Q03JI6.1 (SEQ ID NO:55). In embodiments, the unmodified CRISPR enzyme has DNA cleavage activity, such as Cas9. In embodiments, the CRISPR enzyme is Cas9, and may be Cas9 from S. pyogenes or S. pneumoniae. In embodiments, the CRISPR enzyme directs cleavage of one or both strands at the location of a target sequence, such as within the target sequence and/or within the complement of the target sequence. In embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In embodiments, a vector encodes a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A, where the amino acid numbering is as shown in SEQ ID NO: 1) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N₈₅₄A, and N₈₆₃A (where the amino acid numbering is as shown in SEQ ID NO:19). In embodiments, nickases may be used for genome editing via homologous recombination.

In embodiments, a Cas9 nickase may be used in combination with guide sequence(s), e.g., two guide sequences, which target respectively sense and antisense strands of the DNA target. This combination allows both strands to be nicked and used to induce NHEJ.

In embodiments, genetic manipulation is achieved using a base-editing protein. In embodiments, a base-editing protein is a modified protein (such as a Cas protein or another protein) that catalyzes transitions and/or transversions of one base into another (e.g., A to T, C to G, etc.) without the introduction of a double stranded DNA break. Non-limiting descriptions of such systems are provided in Hess et al. (2016) Directed evolution using dCas9-targeted somatic hypermutation in mammalian cells, Nat Methods. 13(12):1036-1042; Gaudelli et al. (2017) Programmable base editing of A•T to G•C in DNA without DNA cleavage, Nature Volume 551, pages 464-471; Zong et al. (2017) Precise base editing in rice, wheat and maize with a Cas9-cytidine deaminase fusion, Nat Biotechnol. 35(5):438-440; Gehrke et al. (2018) High-precision CRISPR-Cas9 base editors with minimized bystander and off-target mutations, bioRxiv 273938; doi: doi.org/10.1101/273938; and Eid et al. (2018) CRISPR base editors: genome editing without double-stranded breaks, Biochem J. 475(11): 1955-1964, the entire contents of each of which are incorporate herein by reference. In embodiments, the base-editing protein is a base editor that mediates the conversion of A•T to G•C in DNA. In embodiments, the base-editing protein is a base editor that mediates the conversion of C•G to T•A in DNA. In embodiments, the base editor is a Cpf1 base editor. A non-limiting description of a Cpf1 base editor is provided in Li et al. (2018) Base editing with a Cpf1-dytidine deaminase fusion, Nat. Biotechnol. 36(4):324-27.

In embodiments, an RNA-guided protein is fused to a subcellular localization signal (such as a mitochondrial localization signal) to produce an RNA-guided fusion protein. In embodiments, the fusion protein contains a mitochondrial localization signal. Depending on context, an RNA-guided fusion protein comprising, e.g., Cas9 or Cpf1 (or a variant thereof) and a mitochondrial localization signal may be referred to herein (e.g., as “Cas9” or “Cpf1”) without specifying the inclusion of the mitochondrial localization signal. In embodiments, the localization signal is at the N-terminal end of the RNA-guided fusion protein. In embodiments, the localization signal is at the C-terminal end of the RNA-guided fusion protein. A non-limiting example of a mitochondrial localization signal includes MLSLRQSIRFFKPATRTLCSSRYLL (SEQ ID NO:24).

In embodiments, an enzyme coding sequence encoding a CRISPR enzyme is codon optimized for expression in particular cells, such as mammalian cells, e.g., human cells.

In general, and in the context of a CRISPR system, a guide sequence is any polynucleotide sequence having sufficient complementarity with a target polynucleotide sequence to hybridize with the target sequence and direct sequence-specific binding of a CRISPR complex to the target sequence. In embodiments, the degree of complementarity between a guide sequence and its corresponding target sequence, when optimally aligned using a suitable alignment algorithm, is about or more than about 90%, 95%, 97.5%, 98%, 99%, or more. In embodiments, the degree of complementarity is 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). Other useful alignment algorithms are disclosed herein. In embodiments, a guide sequence is about or more than about 15, 16, 17, 18, 19, 20, 25, 30, 35, 40, 45, 50 or more nucleotides in length. In embodiments, a guide sequence is less than about 90, 80, 70, or 60 nucleotides in length.

In embodiments, a target sequence is unique in a mammalian cell (e.g., a human cell). In embodiments, a target sequence is unique in a mitochondria. In embodiments, a target sequence is unique in a polynucleotide (such as a DNA or RNA) that occurs within a mitochondria.

### III. METHODS

In an aspect, included herein is a method of reducing the expression of a mitochondrial protein and/or polynucleotide. In embodiments, the method comprises introducing a compound or complex of the present disclosure into a eukaryotic cell comprising the mitochondria.

In an aspect, included herein is a method of altering the sequence of a mitochondrial polynucleotide (e.g., DNA). In embodiments, the method comprises introducing a compound or complex of the present disclosure into a eukaryotic cell comprising the mitochondria.

In an aspect, included herein is a method of altering the sequence or the expression of at least one mitochondrial polynucleotide. In embodiments, the method comprises introducing into a eukaryotic cell an effective amount of a compound or complex described herein.

In embodiments, the method comprises introducing into a eukaryotic cell an RNA-guided protein. In embodiments, the protein is an RNA-guided protein. In embodiments, the RNA-guided protein is an RNA-guided enzyme. In embodiments, the RNA-guided enzyme is an RNA-guided endonuclease enzyme.

In embodiments the RNA-guided protein comprises a mitochondrial localization amino acid sequence covalently attached to N-terminus thereof.

In embodiments, the RNA-guided endonuclease is a Type II or a Type V CRISPR effector endonuclease. In embodiments, the RNA-guided endonuclease enzyme is a Cas9, a Cpf1 (also known as Cas12a), or a variant thereof. Non-limiting examples of Type II CRISPR endonucleases include Streptococcus pyogenes Cas9 (SpCas9), Staphylococcus aureus Cas9 (SaCas9), and Neisseria meningitides Cas9 (NmCas9). Non-limiting examples of Type V CRISPR endonucleases include Lachnospiraceae bacterium (LbAsCpf1), and Acidaminococcus Cpf1 (AsCpf1).

In embodiments, the Cpf1 is from an Acidaminococcus sp. BV3L6 or Lachnospiraceae bacterium ND2006 (AsCpf1 and LbCpf1, respectively). In embodiments, the Cas9 is Strepyogenes Cas9 (Sp Cas9) or Staphylococcus aureus Cas9 (SaCas9).

In embodiments, the RNA-guided protein comprises a mitochondrial localization amino acid sequence covalently attached to N-terminus of said RNA-guided endonuclease enzyme.

In embodiments, the RNA-guided endonuclease enzyme is a base-editor.

In an aspect, included herein is a method of treating a mitochondrial disorder in a subject in need thereof. In embodiments, the method comprises administering to the subject an effective amount of a compound or complex described herein.

In embodiments, the method comprises introducing into a eukaryotic cell an RNA-guided protein. In embodiments, the protein is an RNA-guided protein. In embodiments, the RNA-guided protein is an RNA-guided enzyme. In embodiments, the RNA-guided enzyme is an RNA-guided endonuclease enzyme.

In embodiments the RNA-guided protein comprises a mitochondrial localization amino acid sequence covalently attached to N-terminus thereof.

In embodiments, the RNA-guided endonuclease enzyme is Cas9, Cpf1, a Class II CRISPR endonuclease or a variant thereof.

In embodiments, the Cpf1 is from an Acidaminococcus sp. BV3L6 or Lachnospiraceae bacterium ND2006 (AsCpf1 and LbCpf1, respectively). In embodiments, the Cas9 is Strepyogenes Cas9 (Sp Cas9).

In embodiments, the RNA-guided protein comprises a mitochondrial localization amino acid sequence covalently attached to N-terminus of said RNA-guided endonuclease enzyme.

In embodiments, the RNA-guided endonuclease enzyme is Cas9, Cpf1, a Class II CRISPR endonuclease or a variant thereof.

In embodiments, the RNA-guided endonuclease enzyme is a base-editor.

In embodiments, the mitochondrial disorder is myoclonic epilepsy with ragged red fibers (MERRF); mitochondrial myopathy, encephalopathy, lactacidosis, and stroke (MELAS); maternally inherited diabetes and deafness (MIDD); Leber's hereditary optic neuropathy (LHON); chronic progressive external ophthalmoplegia (CPEO); Leigh disease; Kearns-Sayre syndrome (KSS); Friedreich's Ataxia (FRDA); co-enzyme QlO (CoQlO) deficiency; complex I deficiency; complex II deficiency; complex III deficiency; complex IV deficiency; complex V deficiency; myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; or cancer.

In embodiments, the compound or complex is in a composition comprising a pharmaceutically acceptable excipient.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A variety of suitable methods for introducing a compound or complex of the present disclosure are available, and generally involve delivering the compound or complex into the cell. In embodiments, the compound or complex is in or complexed with a carrier, such as in a liposome, in a virus, or complexed with a transfection reagent (e.g., a cationic polymer). In embodiments, a compound or complex provided herein is delivered into a cell via electroporation. In embodiments, a compound or complex provided herein is delivered into a cell via a process comprising temporarily deforming a cell as it passes through a small opening to disrupt the cell membrane thereof, and allowing the compound or complex to be inserted into the cell. In embodiments, a compound or complex provided herein is delivered into a cell with a liposome. In embodiments, a compound provided herein is delivered into a cell (e.g., via electroporation, temporary cell deformation) and an RNA-guided protein is expressed in the cell (e.g., from a viral vector or a plasmid).

### IV. EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1. Nucleic Acid Delivery to Mitochondria by Cationic Compounds

Mitochondria are unique dynamic organelles that provide energy for the cell in the form of ATP and carry genomic content. Mitochondrial DNA (mtDNA) encodes for critical subunits in the electron transport chain, and mutations in mtDNA have devastating bioenergetic defects resulting in neuromuscular diseases. Gene therapy approaches aimed at correcting the mutated gene have been limited by the challenges of transforming mtDNA. Alternatively, several endonucleases, including TALENs and zinc-finger nucleases (ZFNs) have been targeted to the mitochondrial matrix to generate double-stranded DNA (dsDNA) breaks in mutated mtDNA and reduce heteroplasmic mutation load via elimination of linearized mtDNA. Implementations of the present subject matter adapt the type V CRISPR system to the mitochondria as a genetic therapy for reducing heteroplasmic mutation load and rescuing bioenergetic defects.

We have targeted the Cpf1 RNA-dependent endonuclease to the mitochondrial matrix using the cytochrome c oxidase subunit 8 (COX8) targeting signal. The amino acid sequence of this targeting signal was MSVLTPLLLRGLTGSARRLPVPRAKIHSL (SEQ ID NO:25). The nucleic acid sequence encoding COX8 was ATGTCCGTCCTGACGCCGCTGCTGCTGCGGGGCTTGACAGGCTCGGCCCGGCGGCTC CCAGTGCCGCGCGCCAAGATCCATTCGTTG (SEQ ID NO:26). To deliver the crRNA required for Cpf1 function, we have attached the RNA to a cyanine compound. Cyanine compounds are cationic lipophilic molecules that accumulate in mitochondria based on the mitochondrial membrane potential. The crRNA accumulates in mitochondria within 48 h of transfection. The RNA import is reversible when mitochondrial membrane potential is dissipated by formalin fixation or addition of an uncoupler.

Mitochondria are unique organelles that are the powerhouse of the cell and carry its own genomic content. Mitochondrial DNA (mtDNA) is a double-stranded circular molecule that encodes 37 genes, 24 of which are necessary for mtDNA translation (2 ribosomal RNAs, 22 transfer RNAs) and 13 subunits of the respiratory chain (complex I, III, IV and V) critical for producing energy in the form of ATP. Mitochondrial DNA is present in hundreds to thousands of copies inside the cell and nucleotide polymorphisms produce a state of heteroplasmy. A high heteroplasmic load of mutation can cause a bioenergetics defects, cellular damage from reactive oxygen species, and trigger cell death. Many mitochondrial diseases lead to devastating disorders of encephalomyopathies wherein tissues with high metabolic demands, such as musculoskeletal and neuronal tissues, are severely affected.

Strategies aimed at eliminating mutant mtDNA have shown to be effective in shifting heteroplasmy towards lower mutation load and rescuing cellular metabolic defects (1). There are limited DNA repair mechanisms in mammalian mitochondria. Given the high redundancy of mitochondrial genome in the cell, clearance of mtDNA is a predominant mechanism in protecting the fidelity of mtDNA in mammalian cells (2, 3). As a result, targeting of restriction endonucleases or homing endonucleases, such as transcription activator-like effector nucleases (TALENs) and zinc-finger nucleases (ZFNs), to mitochondria with high levels of heteroplasmy resulted in successful depletion of mutant mtDNA and rescue of metabolic defects (2, 4-7). Furthermore, these gene editing modalities have been utilized for editing heteroplasmy in the germline of murine models and human oocytes with minimal adverse effects on development of the cell or the fitness of the animal suggesting that the strategy is a viable clinical therapy (8). However, the generalizability of these tools for clinical therapy is limited. Usage of restriction endonucleases requires a specific mutation that creates a compatible restriction site and thus it is not a generalizable technique for the vast amount of characterized mtDNA mutations. Homing endonucleases such as TALENs and ZFNs require mitochondrial import of large bulky protein motifs for sequence recognition. The large sizes of the DNA binding motifs can be difficult to produce, result in insufficient expression and poor localization in mitochondria. Although mitochondrial replacement by means of mitochondrial in vitro fertilization (IVF), wherein the nucleus of patient's oocyte is transplanted into enucleated oocyte from a healthy mitochondrial donor, is a new therapy, it is only approved in the United Kingdom.

Herein we propose to adapt the genome editing technology known as CRISPR, clustered regularly interspaced short palindromic repeat, towards manipulating mtDNA. Since the first demonstration that Cas9 protein can be engineered to cleave specific DNA sequences in 2013, the CRISPR-Cas9 technology has been transformative in the research community by simplifying genome editing in many cell types and animal models. The class II CRISPR system is a genome editing technology derived from bacteria and archaea that utilizes a single guide RNA (sgRNA), or a crRNA and tracrRNA complex, to direct a single effector endonuclease to cleave specific DNA sequences. The Cpf1 endonuclease is a novel class II type V system that is distinct from the Cas9 system in several features. Firstly, Cpf1 is a smaller endonuclease that utilizes a T-rich PAM domain located at the 5′ end of the non-target DNA in contrast to Cas9 protein that relies on a G-rich PAM site at the 3′ end of the non-target DNA strand (9). Thus, the AT rich genome of mitochondria is better suited for gene editing using the Cpf1 system. Secondly, Cpf1 introduces a staggered double-stranded break with a 4 to 5 nucleotide overhang (10). Thirdly, the double stranded break occurs at the 3′ end of the guide RNA and thus preserves the PAM recognition domain for potentially subsequent cleavage. And lastly, Cpf1 does not require a tracr RNA element resulting in a shorter guide RNA that contains a crRNA followed by a spacer domain targeting the DNA of interest. Recent studies have demonstrated that the length of the spacer domain can be further truncated from 23 nt to 19-21 nt without significant effects on cleavage activity of Cpf1 (9, 11).

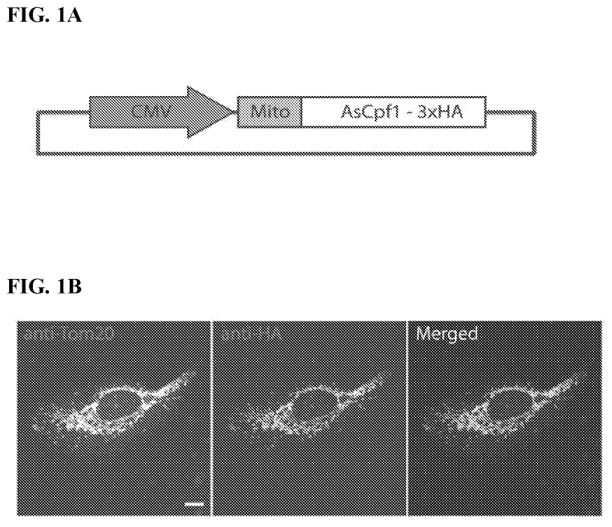

Cpf1 from both Acidaminococcus sp. BV3L6 and Lachnospiraceae bacterium ND2006 (AsCpf1 and LbCpf1, respectively) species have demonstrated efficient genome-editing in human cells comparable to Strepyogenes Cas9 (Sp Cas9) (10, 11). We have constructed a mitochondrially targeted AsCpf1 using Cox8 targeting signal. There is an HA tag to track the localization of AsCpf1 and the construct is driven by a CMV promoter (FIG. 1A). Immunofluorescence studies show that AsCpf1 co-localizes with Tom20, a mitochondrial membrane protein (FIG. 1B). There is a Neomycin cassette in the plasmid to enable selection of colonies stably expressing the construct. We have established clonal population of HeLa cells with stable expression of mitochondrially targeted AsCpf1 (mitoAsCpf1) and will be using these cultures to determine cleavage activity of Cpf1.

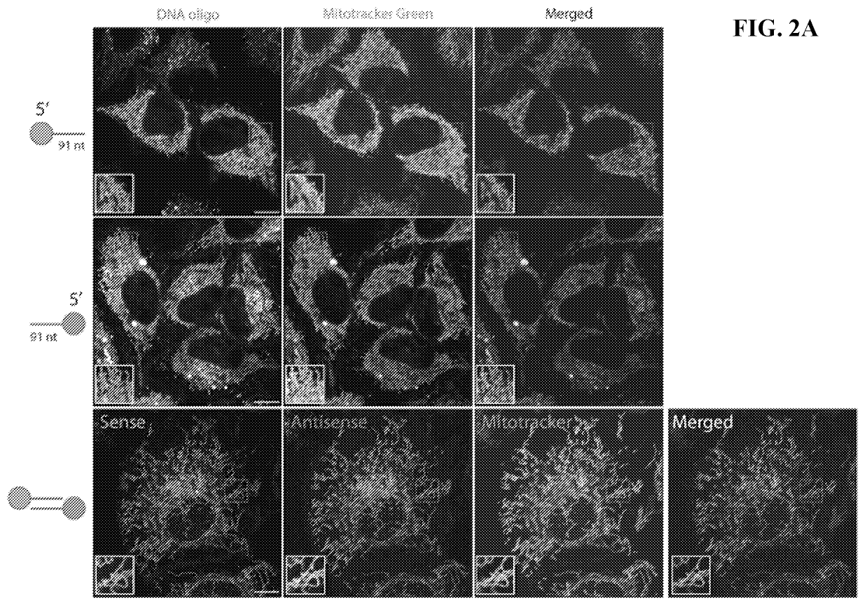

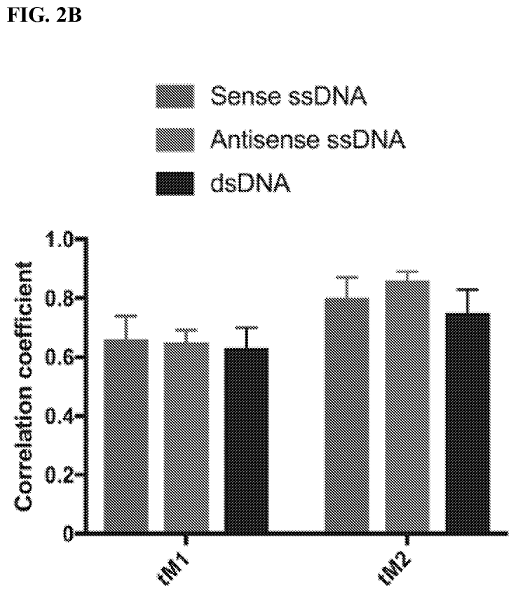

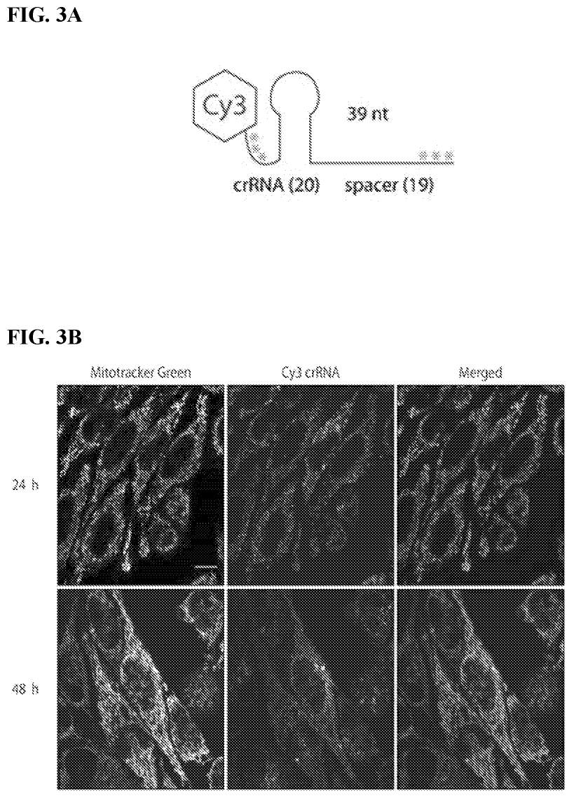

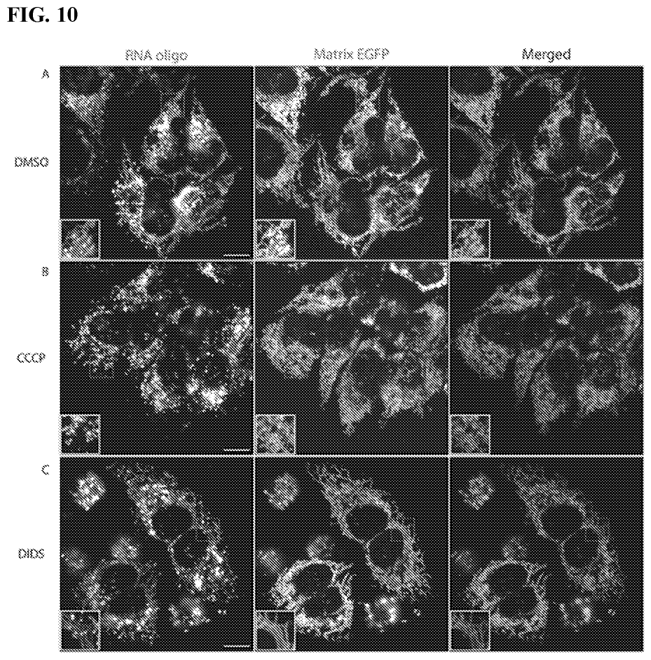

We have identified that Cy3 or Cy5 can deliver a variety of single stranded oligonucleotides and modified RNA sequences to the mitochondria, including separately labeled complementary oligonucleotides as double-stranded linear DNA (see, e.g., FIG. 2). We achieved mitochondrial localization of AsCpf1 crRNA with a 19 or 21 nt spacer domain (see, e.g., FIG. 3). It is important to note that cationic molecules are selectively concentrated in mitochondria as a result of intact mitochondrial membrane potential generated across the inner membrane. We have confirmed that dissipation of the mitochondrial membrane potential, using uncoupling reagents, such as carbonyl cyanide m-chlorophenyl hydrazone (CCCP) and formalin, results in immediate release of Cy3-RNA from mitochondria (see, e.g., FIG. 10).

The cyanine dyes were the only tested dyes that successfully showed mitochondrial import. Specifically, we have tested Cy3 and Cy5, which both worked, indicating that other cyanine dyes will also work, based on their chemical similarities. We also tried ATTO 647N and FAM dyes, neither of which worked. It is surprising that the ATTO 647N dye did not work, as it carries a strong positive charge, akin to the cyanine dyes. Without being bound by scientific theory, it is possible that a feature of cyanine compounds, other than or in addition to the charge thereof, facilitates mitochondrial import of linked polynucleotides.

While a 3′-cyanine moiety was found not to facilitate mitochondrial transport on its own, a 3′-cyanine does not inhibit mitochondrial localization on a polynucleotide that is labeled at both the 5′ and 3′ ends with a cyanine moiety. An experiment with a polynucleotide carrying a 5′ Cy5 and a 3′ Cy 3 showed that it successfully localized to the mitochondria.

RNA polynucleotides are highly susceptible to degradation by cellular RNases in the cytoplasm, so the stability of RNA in the cytoplasm is likely to be less than of DNA. This is important since, in embodiments, polynucleotides transition from the cytoplasm to the mitochondria. For this reason, we anticipate greater efficiency of DNA import from the cytoplasm to the mitochondrial matrix than for unmodified RNA. To mitigate degradation, synthetic RNA is generally stabilized with chemical substitution of the ribose ring or the phosphodiester backbone. The most common substitutions include replacement of the 2′-hydroxy (2′-OH) group with a 2′-fluoro (2′-F) or a 2′-O-methyl (2′-OMe) or replacement of the phosphodiester backbone with phosphorothioate (PS) linkers. We have tested all of these different modifications and found that each stabilizes the RNA polynucleotides relative to unmodified RNA, but all of these modifications do not necessarily enhance mitochondrial localization, potentially due to the electrostatic charge differences between each of these modifications (more on this below).

Without being bound by any scientific theory, we hypothesize that cyanide dyes are functional as mitochondrial transporters of polynucleotides due to their high positive charge. Due to the 2′-OH group characteristic of RNA, RNA has a stronger negative charge than DNA, so the net-positive charge of a Cy3/Cy5-labeled RNA oligo is lower than the net-positive charge of a Cy3/Cy5-labeled DNA oligo. Thus, we expect Cy3/Cy5-labeled RNA to be imported less efficiently to mitochondria than Cy3/Cy5-labeled DNA.

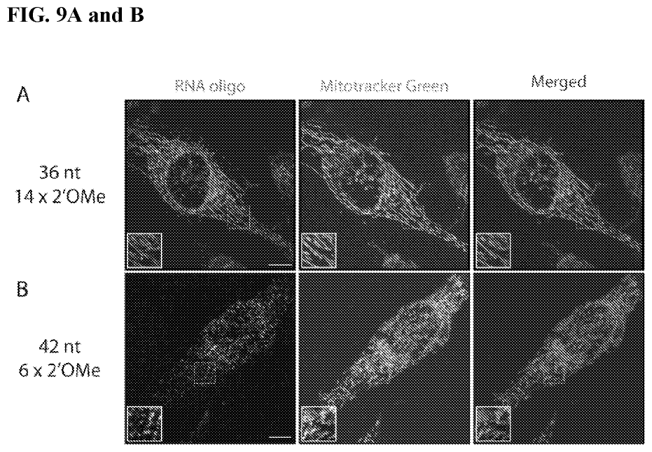

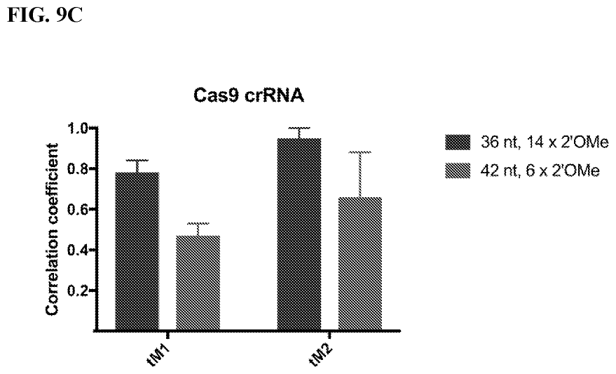

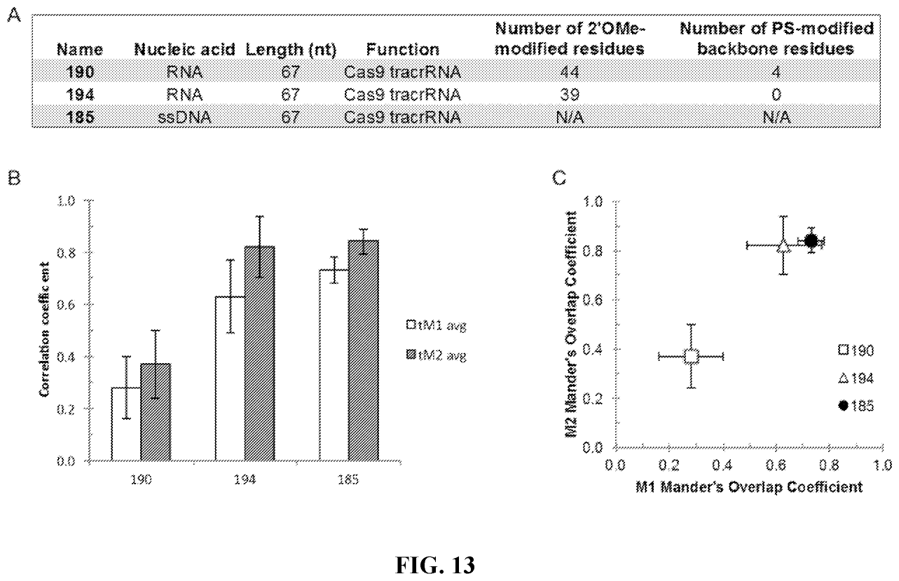

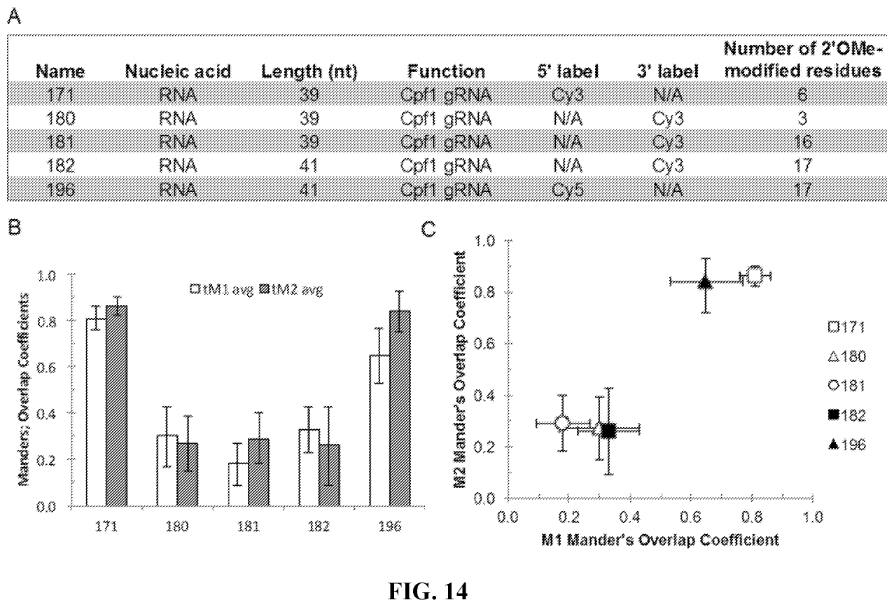

This notion is consistent with our experimental observations. Specifically, we have tested both ssDNA and RNA oligos of the same length and sequence to observe that both long (e.g., >90 nt) oligos localize to mitochondria with high efficiency (see, e.g., FIG. 13). Substitution of one or more RNA nucleic acid residues with 2′O-methyl residues, which generally reduces negative charge, appears to improve mitochondrial localization (see, e.g., FIG. 9C). Without wishing to be bound by theory, the improvement in mitochondrial localization with 2′O-methyl residues may be due to charge-based properties, resistance to nucleolytic degradation, or a combination of these. However, substitution of the 2′-OH with 2′-fluoro residues does not improve mitochondrial localization, even though the 2′-F and 2′-OMe modifications similarly stabilize the oligo from degradation (see, e.g., FIG. 8, Row C). Without being bound by any scientific theory, this is likely due to the strong electronegative charge of the fluoro atoms, which increase the overall negative charge of the oligo. Likewise, substitution of the phosphodiester backbone with phosphorothioate (PS) (which makes the oligo more polianionic) reduces its mitochondrial localization (see, e.g., FIG. 8, Row B; and FIG. 13). It is notable that the commercially sold tracrRNA from IDT is modified with both 2′-O-methyl and PS substitutions. We have found that this molecule does not efficiently localize to mitochondria after 5′-end-labeling with Cy3. Only when we removed the PS substitutions were we able to achieve mitochondrial localization (see, e.g., FIG. 13).

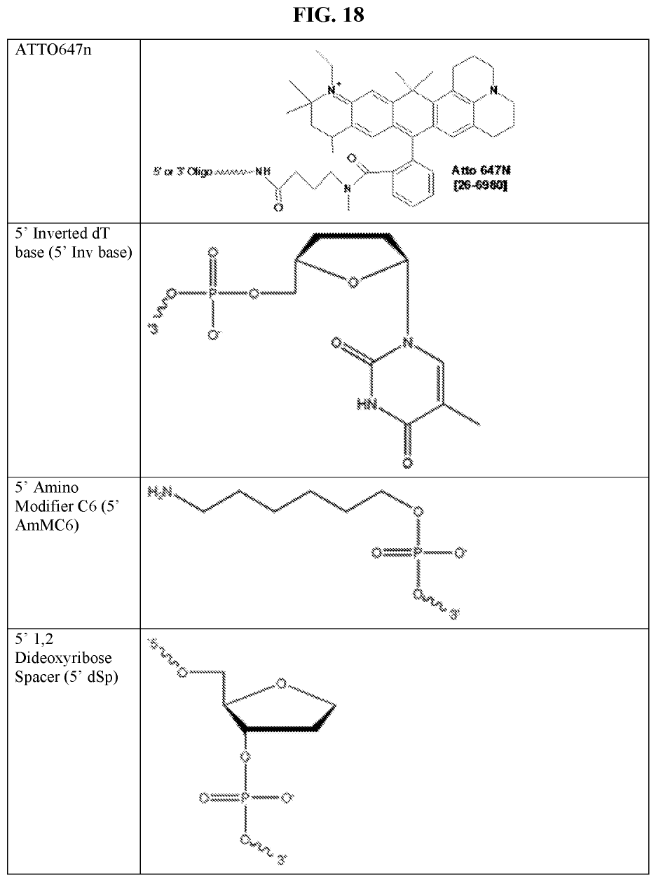

We have observed that DNA or modified RNA with a terminal 3′ Cy3 does not successfully localize in the mitochondria (unless there was also a 5′ cyanine moiety). Rather, the fluorescent signal was very weak, and mostly concentrated in vesicles (perhaps endosomes or lysosomes). This suggests that the import mechanism of these polynucleotides has a directionality from a 5′ moiety on the polynucleotide. We also observed a significant difference in the patterns of RNA molecules with 2′O-methyl residues versus similar RNA molecules with 2′-fluoro residues. Generally speaking, these two types of modifications each prevent degradation from enzymatic degradation, and both are used for other types of RNA therapeutics (e.g., RNA aptamers, antisense oligos). However, the 2′-fluoro group has a very strong negative charge, whereas the 2′-O-methyl is more neutral. We observed that the 2′-fluoro modified RNA molecules did not degrade, but did become trapped in intracellular vesicles and did not localize to the mitochondrial matrix (see, e.g., FIG. 8, Rows A and C). This is an important experiment, as it shows that increasing the net negative charge of the molecule with 2′-fluoro appears to prevent mitochondrial localization, but reducing the net negative charge with 2′-O-methyl appears to improve mitochondrial localization. However, fewer 2′-F substitutions might not prevent mitochondrial localization. Additional details regarding tested oligonucleotide sequences are presented in Table 1, in which “mX” designates a nucleotide having a 2′OMe modification, and “rX” designates a ribonucleotide. Structures of modifications that did not efficiently direct transport to the mitochondria are depicted in FIG. 18.

The cytoplasm is rich in RNA-binding proteins, whereas DNA-binding proteins are mostly found in the nucleus, where they bind to genomic DNA. Thus, it is much more likely that cellular cytoplasmic RNA-binding proteins can bind and sequester RNA polynucleotides in the cytoplasm, thereby preventing their localization to mitochondria. This likelihood is much less for DNA, since DNA-binding proteins (e.g., histones, transcription factors, DNA enhancer proteins) are generally not found in the cytoplasm. The natural affinity for RNAs to cytoplasmic RNA-binding proteins can lead to the sequestration of RNAs in the cytoplasm and thus prevent migration to the mitochondria, whereas DNA-binding proteins are abundant in the nucleus but not the cytoplasm and do not alter the mitochondrial localization of DNA polynucleotides.

Thus, three reasons that we believe are particularly important in differentiating DNA from RNA polynucleotides for mitochondrial localization are differences in charge, stability, and motility.

TABLE 1
Cy_oligo sequences tested

| | Oligo | 5′ | Length | | | Endo- | tM1 | st | tM2 | St | |
|---|---|---|---|---|---|---|---|---|---|---|---|
| DNA/RNA | Name | or 3′ | (nt) | Label | Target | nuclease | avg | dev | avg | dev | Sequence |
| RNA * | 152 | 5′ | 42 | Cy3 | HSP | Cas9 | 0.66 | 0.10 | 0.79 | 0.09 | 5′Cy3/mUmGmGrGrGrGrGr |
| | | | | | | crRNA | | | | | UrGrUrCrUrUrUrGrGrGrG |
| | | | | | | | | | | | rUrUrGrUrUrUrUrArGrAr |
| | | | | | | | | | | | GrCrUrArUrGrCrUrGrUrU |
| | | | | | | | | | | | mUmUmG (SEQ ID NO: 1) |
| RNA ** | 171 | 5′ | 39 | Cy3 | HSP | Cpf1, | 0.81 | 0.05 | 0.86 | 0.04 | 5′Cy3/mUmAmArUrUrUrCr |
| | | | | | | 39 nt | | | | | UrArCrUrCrUrUrGrUrArG |
| | | | | | | | | | | | rArUrGrGrGrUrUrUrGrGr |
| | | | | | | | | | | | UrUrGrGrUrUrCrGmGmGmG |
| | | | | | | | | | | | (SEQ ID NO: 2) |
| RNA | 180 | 3′ | 39 | Cy3 | HSP | Cpf1 | 0.30 | 0.13 | 0.27 | 0.12 | rUrArArUrUrUrCrUrArCr |
| | | | | | | | | | | | UrCrUrUrGrUrArGrArUrG |
| | | | | | | | | | | | rGrGrUrUrUrGrGrUrUrGr |
| | | | | | | | | | | | GrUrUrCrGmGmGmG/3′Cy3 |
| | | | | | | | | | | | (SEQ ID NO: 3) |
| RNA | 181 | 3′ | 39 | Cy3 | HSP | Cpf1 | 0.18 | 0.09 | 0.29 | 0.11 | mUrArArUmUmUmCmUmAmCr |
| | | | | | | | | | | | UmCmUmUrGraUmAmGmArUr |
| | | | | | | | | | | | GrGrGrUrUrUrGrGrUrUrG |
| | | | | | | | | | | | rGrUrUrCrGrGmGmG/ |
| | | | | | | | | | | | 3′Cy3 (SEQ ID NO: 4) |
| RNA | 182 | 3′ | 41 | Cy3 | HSP | Cpf1 | 0.33 | 0.10 | 0.26 | 0.17 | mUrArArUmUmUmCmUmAmCr |
| | | | | | | | | | | | UmCmUmUrGmUmAmGmArUrG |
| | | | | | | | | | | | rGrGrUrUrUrGrGrUrUrGr |
| | | | | | | | | | | | GrUrUrCrGrGrGmGmUmA/ |
| | | | | | | | | | | | 3′Cy3 (SEQ ID NO: 5) |
| RNA ** | 188 | 5′ | 36 | Cy3 | 14787 | Cas9 | 0.78 | 0.06 | 0.95 | 0.05 | 5′Cy3/mGmAmGmUrGrGrUr |
| | | | | | del4 | crRNA | | | | | UrArGrUrUrUrUrArUrUrA |
| | | | | | | | | | | | rGrGrGrUrUrUrUrAmGmAm |
| | | | | | | | | | | | GmCmUmAmUmGmCmU |
| | | | | | | | | | | | (SEQ ID NO: 6) |
| RNA | 190 | 5′ | 67 | Cy5 | | Cas9 | 0.28 | 0.12 | 0.37 | 0.13 | 5′mA*mG*mCmAmUmAmGmCm |
| | | | | | | tracrRNA | | | | | AAGUUAAAAUAAGGCUAGUCC |
| | | | | | | | | | | | GUUmAmUmCmAmAmCmUmUmG |
| | | | | | | | | | | | mAmAmAmAmAmGmUmGmGmCm |
| | | | | | | | | | | | AmCmCmGmAmGmUmCmGmGmU |
| | | | | | | | | | | | mGmCmU*mU*mU (SEQ ID |
| | | | | | | | | | | | NO: 7)(IDT tracrRNA |
| | | | | | | | | | | | with 5′ Cy5; |
| | | | | | | | | | | | “*” designates |
| | | | | | | | | | | | phosphorothioate |
| | | | | | | | | | | | bond) |
| ssDNA ** | 193 | 5′ | 41 | 5′ Cy5, | HSP | Cpf1 | 0.65 | 0.10 | 0.79 | 0.10 | 5′Cy5/TAATTTCTACTCTTG |
| | | and 3′ | | 3′ Cy3 | | | | | | | TAGATGGGTTTGGTTGGTTCG |
| | | | | | | | | | | | GGGTA/3′Cy3 |
| | | | | | | | | | | | (SEQ ID NO: 8) |
| RNA ** | 194 | 5′, 39 | 67 | Cy5 | n/a | Cas9 | 0.63 | 0.14 | 0.82 | 0.12 | 5′Cy5/mAmGmCmAmUmAmGm |
| | | OME | | | | tracrRNA | | | | | CmArArGrUrUrArArArArU |
| | | | | | | | | | | | rArArGrGrCrUrArGrUrCr |
| | | | | | | | | | | | CrGrUrUrArUrCrArAmCmU |
| | | | | | | | | | | | mUmGmAmAmAmAmAmGmUmGm |
| | | | | | | | | | | | GmCmAmCmCmGmAmGmUmCmG |
| | | | | | | | | | | | mGmUmGmCmUmUmU |
| | | | | | | | | | | | (SEQ ID NO: 9) |
| RNA ** | 196 | 5′ | 41 | Cy5 | HSP | Cpf1 | 0.65 | 0.12 | 0.84 | 0.09 | 5′Cy5/mUrArArUmUmUmCm |
| | | | | | | | | | | | UmAmCrUmCmUmUrGmUmAmG |
| | | | | | | | | | | | mArUrGrGrGrUrUrUrGrGr |
| | | | | | | | | | | | UrUrGrGrUrUrCrGrGrGmG |
| | | | | | | | | | | | mUmA (SEQ ID NO: 10) |
| RNA ** | 197 | 5′ | 56 | Cy5 | HSP | Cpf1, + | 0.64 | 0.12 | 0.83 | 0.08 | 5′Cy5/mGmUmCmAmAmAmAm |
| | | | | | | pre-crRNA | | | | | GmAmCmCmUmUmUmUmUrArA |
| | | | | | | | | | | | rUmUmUmCmUmAmCrUmCmUm |
| | | | | | | | | | | | UrGmUmAmGmArUrGrGrGrU |
| | | | | | | | | | | | rUrUrGrGrUrUrGrGrUrUr |
| | | | | | | | | | | | CrGrGrGmGmUmA |
| | | | | | | | | | | | (SEQ ID NO: 11) |
| ssDNA ** | 183 | 5′ | 41 | Cy3 | HSP | Cpf1 | 0.74 | 0.09 | 0.86 | 0.11 | 5′Cy3/TAATTTCTACTCTTG |
| | | | | | | | | | | | TAGATGGGTTTGGTTGGTTCG |
| | | | | | | | | | | | GGGTA (SEQ ID NO: 12) |
| ssDNA | 184 | 3′ | 41 | Cy3 | HSP | Cpf1 | 0.37 | 0.08 | 0.36 | 0.14 | TAATTTCTACTCTTGTAGATG |
| | | | | | | | | | | | GGTTTGGTTGGTTCGGGGTA/ |
| | | | | | | | | | | | 3′Cy3 (SEQ ID NO: 13) |
| ssDNA ** | 185 | 5′ | 67 | Cy5 | | Cas9 | 0.73 | 0.05 | 0.84 | 0.05 | 5′Cy5/AGCATAGCAAGTTAA |
| | | | | | | tracrRNA | | | | | AATAAGGCTAGTCCGTTATCA |
| | | | | | | | | | | | ACTGAAAAAGTGGCACCGAGT |
| | | | | | | | | | | | CGGTGCTTT |
| | | | | | | | | | | | (SEQ ID NO: 14) |
| ssDNA | 186 | 5′ | 41 | ATT | HSP | Cpf1 | 0.32 | 0.09 | 0.20 | 0.10 | 5′ATTO647n/TAATTTCTAC |
| | | | | O647n | | | | | | | TCTTGTAGATGGGTTTGGTTG |
| | | | | | | | | | | | GTTCGGGGTA |
| | | | | | | | | | | | (SEQ ID NO: 15) |
| ssDNA | 189 | 3′ + 5′ | 41 | Cy3 | HSP | Cpf1 | 0.25 | 0.12 | 0.31 | 0.14 | TAATTTCTACTCTTGTAGATG |
| | | inv base | | | | | | | | | GGTTTGGTTGGTTCGGGGTA/ |
| | | | | | | | | | | | 3′Cy3 (SEQ ID NO: 16) |
| SSDNA | 191 | 5′ Am | 41 | Cy3 | HSP | Cpf1 | 0.35 | 0.11 | 0.26 | 0.13 | 5AmMC6/TAATTTCTACTCTT |
| | | MC6 | | | | | | | | | GTAGATGGGTTTGGTTGGTTC |
| | | | | | | | | | | | GGGGTA/3′Cy3 |
| | | | | | | | | | | | (SEQ ID NO: 17) |
| SSDNA | 192 | 5′ dSp | 41 | Cy3 | HSP | Cpf1 | 0.30 | 0.11 | 0.18 | 0.17 | 5dSp/TAATTTCTACTCTTGT |
| | | | | | | | | | | | AGATGGGTTTGGTTGGTTCGG |
| | | | | | | | | | | | GGTA/3′Cy3 |
| | | | | | | | | | | | (SEQ ID NO: 18) |
| SSDNA ** | 195 | 5′ | 91 | Cy5 | HSP | | 0.66 | 0.08 | 0.80 | 0.07 | 5′Cy5/GCTGCTAACCCCATA |
| | | | | | promoter | | | | | | CCCCGAACCAACCAAACCCCA |
| | | | | | | | | | | | AAGACAATGGGCAAGCCCATC |
| | | | | | | | | | | | CCCAACCCCTTGCTTGGCTTG |
| | | | | | | | | | | | GACAGCACCTAA |
| | | | | | | | | | | | (SEQ ID NO: 20) |
| ssDNA ** | 196 | 5′ | 91 | Cy3 | HSP | | 0.65 | 0.04 | 0.86 | 0.03 | 5′Cy3/TTAGGTGCTGTCCAA |
| | | | | | promoter | | | | | | GCCAAGCAAGGGGTTGGGGAT |
| | | | | | | | | | | | GGGCTTGCCCATTGTCTTTGG |
| | | | | | | | | | | | GGTTTGGTTGGTTCGGGGTAT |
| | | | | | | | | | | | GGGGTTAGCAGC |
| | | | | | | | | | | | (SEQ ID NO: 21) |
| dsDNA ** | 195- | | 91 | Cy5 & | HSP | | 0.62- | 0.07 | 0.74- | 0.05- | (195 (SEQ ID NO: 20) |
| | 196 | | | C3 | promoter | | 0.64 | | 0.77 | 0.08 | annealed to 196 |
| | | | | | | | | | | | (SEQ ID NO: 21)) |

*Colocalized with small punctate mitochondria.
**Colocalization positive. 189, 191, 192 with 5′ modifications that did not lead to colocalization.

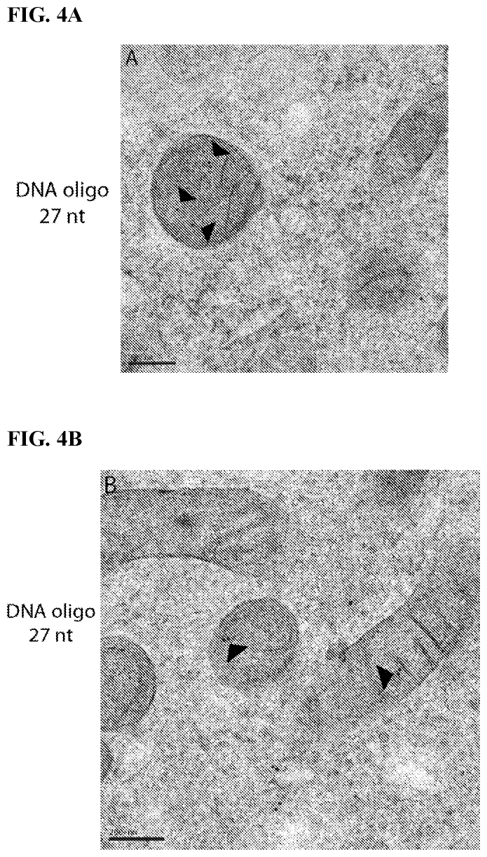

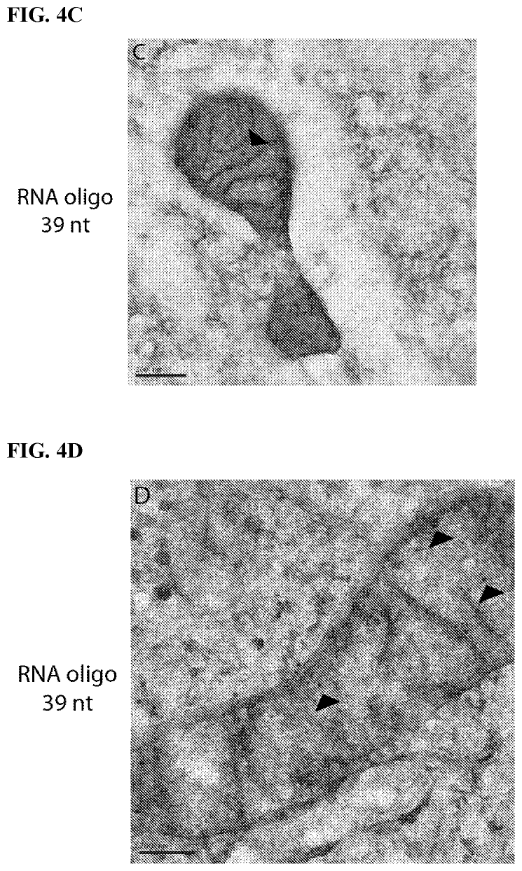

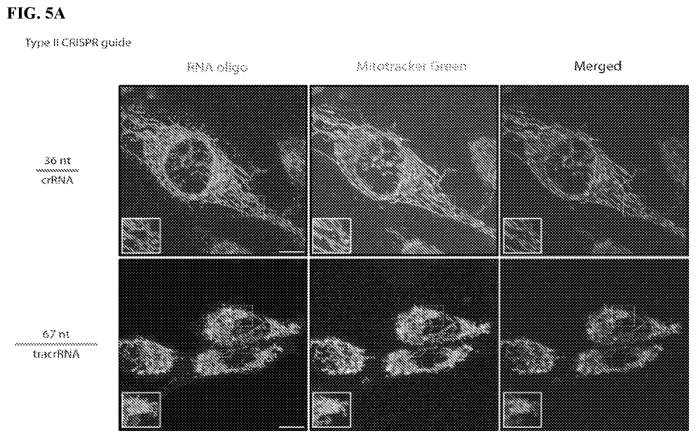

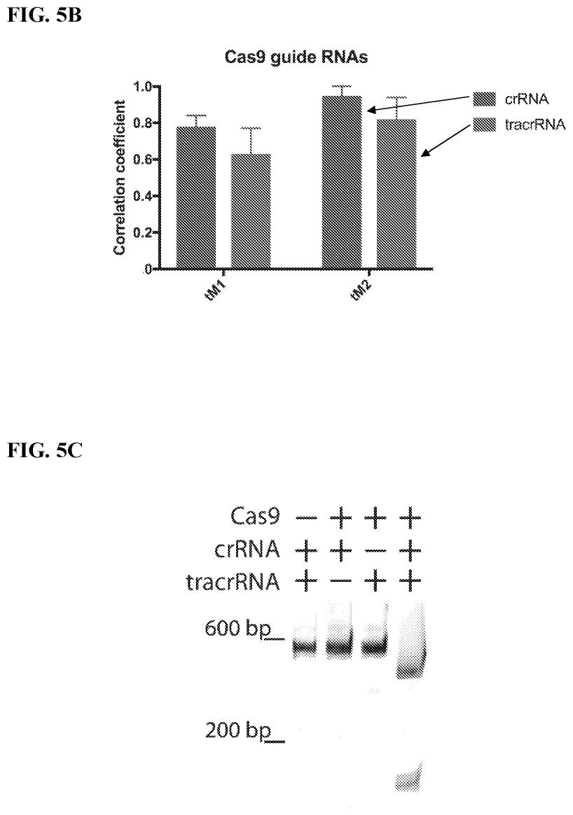

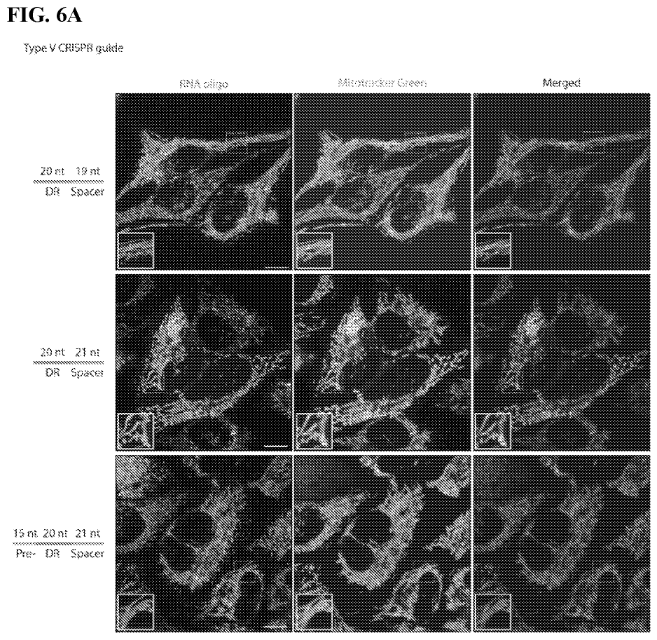

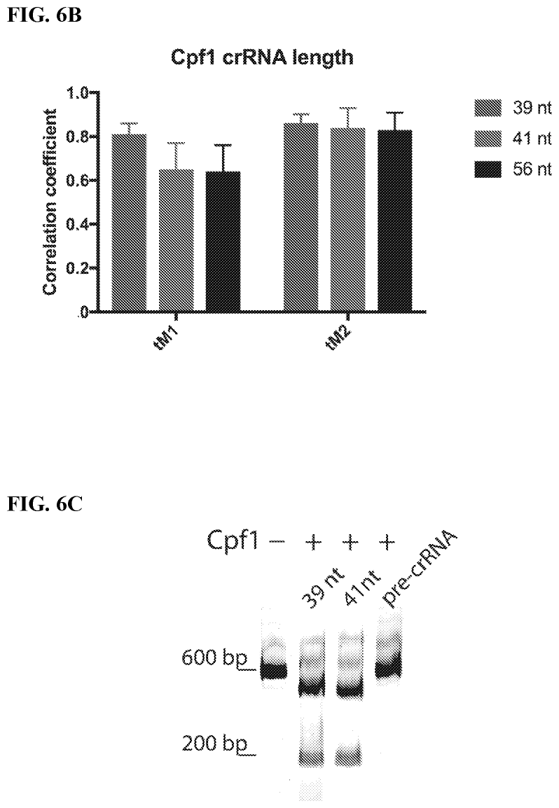

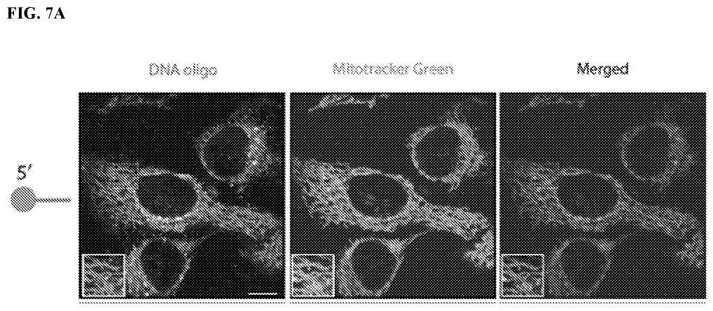

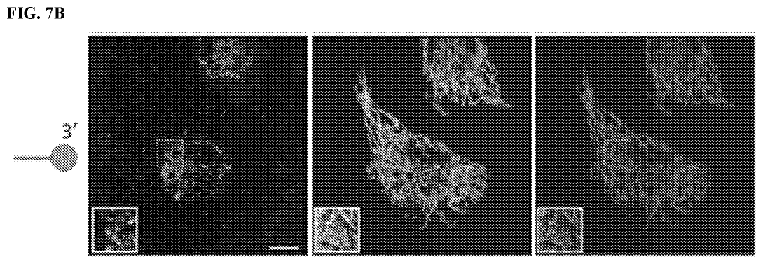

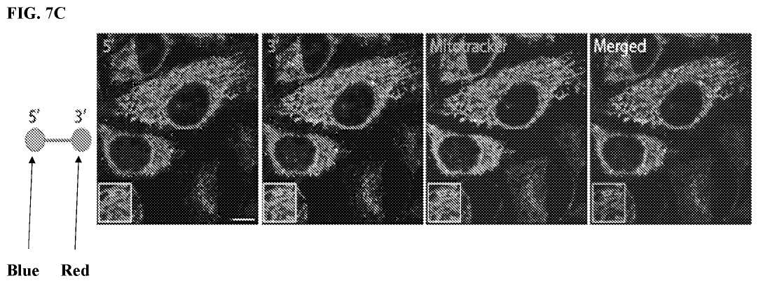

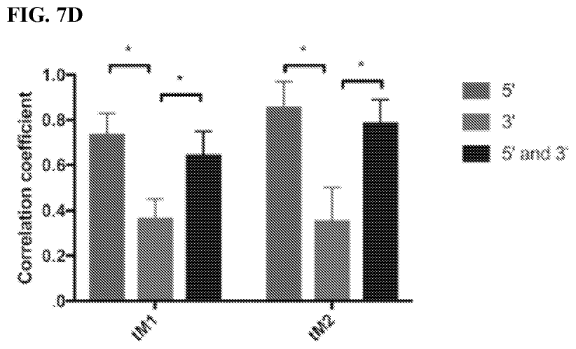

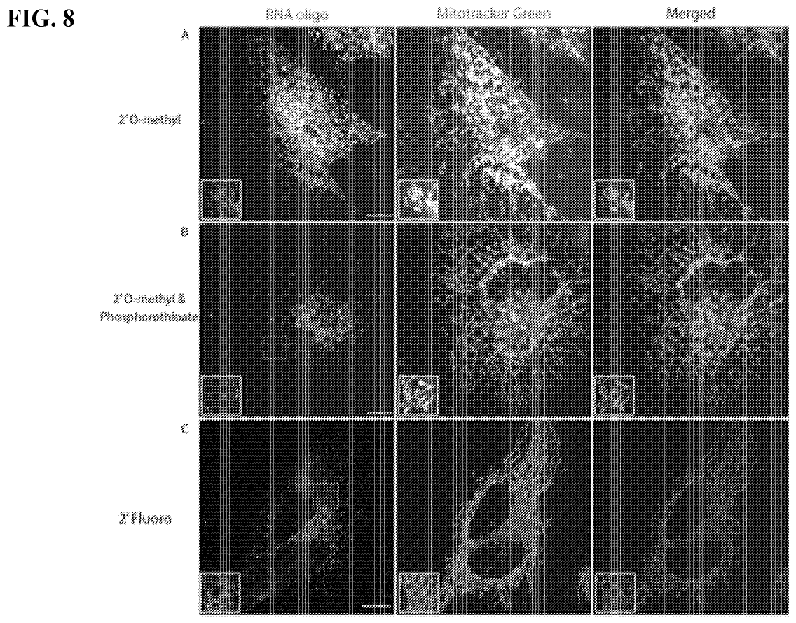

### Example 2. Methods for FIGS. 4-7 Directed to CRISPR Experimentation

In vitro cutting assay. For the type II CRISPR system, 500 nM of each crRNA and tracRNA were assembled with 50 nM of purified Cas9 protein (NEB) in cleavage buffer (NEB). Ribonucleoprotein complex formation occurred at room temperature for 10 min. The target DNA was obtained by PCR amplification and purified by phenol chloroform followed by ethanol precipitation. A total of 22 nM of DNA target was added to the reaction and incubated at 37° C. for 1 hour. The final cleavage products were ran on 3% low melting agarose gel or 6% denaturing TBE polyacrylamide gel. For the type V CRISPR system, 5 μM of crRNA was complexed with 1 μM of Cpf1 endonuclease (IDT Alt-R) and 20 nM of DNA target in cleavage buffer containing 20 mM Tris pH7.6 and 50 mM KCl. The reaction was incubated at 37° C. overnight and products ran on 6% denaturing TBE polyacrylamide gel.

Imaging and analysis. HeLa cells were imaged live using Zeiss 880 LSM confocal microscope with Airy scanner under a heated stage with 5% CO2 incubation. Image acquisition utilized the super-resolution capabilities of the Airy scanner. The Cy3 dye was excited by the 561 nm laser while the Cy5 dye was excited by the 633 nm laser. The Mander's correlation coefficient was calculated using the Colocalization Analysis and Coloc2 plugins in ImageJ (NIH). The Mitotracker Green channel is used as the ROI/mask to quantify the signal of DNA or RNA oligos.

Electroporation of CRISPR endonuclease and guide RNAs. Electroporation of Hela cells was performed according to the Amaxa® Nucleofector® Kit R. Briefly, 1×10E06 Hela cells were resuspended in 100 μl Nucleofector solution and supplement (at ratio of 4.5:1). A total of 5 μg of plasmid DNA expressing the endonuclease and 250-500 pmol of crRNA and 500 pmol of tracrRNA were added to the cells. The electroporation settings for Hela cells were selected in the Amaxa electroporator. Cells were recovered in pre-warmed DMEM with 10% FBS, 1% penicillin-streptomycin and 50 μg/mL of uridine and 2 mM GlutaMAX™. Cells were cultured in humidified 37° C. incubator with 5% CO₂. Media was replaced every other day until cells were collected for mtDNA analysis.

mtDNA purification. Purification of mtDNA utilized the organic solvent extraction method described by Guo W. et al. 2009 Mitochondrion. Briefly, cells were frozen at −20° C. for 1 hour prior to the addition of lysis buffer (10 mM Tris-HCl pH 8, 1 mM EDTA, 0.1% SDS and 1× proteinase K). Cell were lysed by incubation in a 55° C. water bath overnight. Cell lysates were briefly centrifuged for 5 min at maximum speed to remove non-soluble fraction. The lysate was transferred to a new tube containing 1:1 volume ratio of phenol/chloroform/isoamyl alcohol (25:24:1) pH 8. Samples were mixed vigorously and centrifuged at max speed for 15 min. The upper aqueous layer was transferred to a new tube containing 1:1 volume ratio of chloroform. Samples were then mixed vigorously and centrifuged at max speed for 10 min. The upper aqueous layer was transferred to a new tube. The mtDNA was precipitated by adding 0.1 volume of 3M sodium acetate pH 5.2 and 1 volume of isopropanol and incubating at −20° C. for 10 min. The DNA precipitate was collected after centrifugation at max speed for 10 min, washed with 1 ml of 70% ethanol, air dried, and dissolved in TE.

Multiplex Taqman qPCR. All quantitative qPCR assays were performed using iQ™ Multiplex Powermix (Bio-Rad) on the CFX96™ thermocycler (Bio-Rad). Measurements were performed in triplicates. Each reaction contained 25-50 ng of DNA, 250 nM of each primer, 200 nM of each probe in a total volume of 20 μl. The following thermal parameters consist of 95° C. for 15 min and 40 cycles of 95° C. for 15 sec, 65° C. for 20 sec, and 72° C. for 20 sec. Plasmid standards were created for cytochrome B, β-actin, and Woodchuck Hepatitis Virus post-transcriptional regulatory element (WPRE) to assess copy number of mtDNA, nuclear DNA, and endonuclease, respectively. Serial dilutions of standards were performed to assess the linearity of the assay conditions. mtDNA copy number was normalized to β-actin as a measure of mtDNA content per cell. Primers and probes used in detecting the indicated targets are provide in Table 2 below:

TABLE 2

| Target | Primers | Sequence | Probe |
|---|---|---|---|
| β-actin | AP083 | ACCTGACTGACTACC | 5′-Cy5/AGCG |
| | JB_ | TCATGAAGATCCTCA | GGAAATCGTGC |
| | bactin_F | CCGA | GTGACATTAAG |
| | | (SEQ ID NO: 27) | G/Black |
| | AP084 | GGAGCTGGAAGCAGC | hole |
| | JB_ | CGTGGCCATCTCTTG | quencher/-3′ |
| | bactin_R | CTCGAA | (SEQ ID |
| | | (SEQ ID NO: 28) | NO: 29) |
| Cyto- | AP066 | GCCTATATTACGGAT | 5′-HEX/CCTG |
| chrome | cytB_F | CATTTCTCTACT | AAACATCGGCA |
| B | | (SEQ ID NO: 30) | TTATCCTCCTG |
| | AP067 | GCCTATGAAGGCTGT | CT/Black |
| | cytB_R | TGCTATAGT | hole |
| | | (SEQ ID NO: 31) | quencher/-3′ |
| | | | (SEQ ID |
| | | | NO: 32) |
| WPRE | AP173 | GGCCCGTTGTCAGGC | 5′-FAM/TGCT |
| | WPRE_F | AACGTGG | GACGCAACCCC |
| | | (SEQ ID NO: 33) | CACTGGT/ |
| | AP174 | GGAAAGGAGCTGACA | Black hole |
| | WPRE_R | GGTGGTGGCAATG | quencher/-3′ |
| | | (SEQ ID NO: 34) | (SEQ ID |
| | | | NO: 35) |

### Example 3: CRISPR-Directed Depletion of Mitochondrial DNA

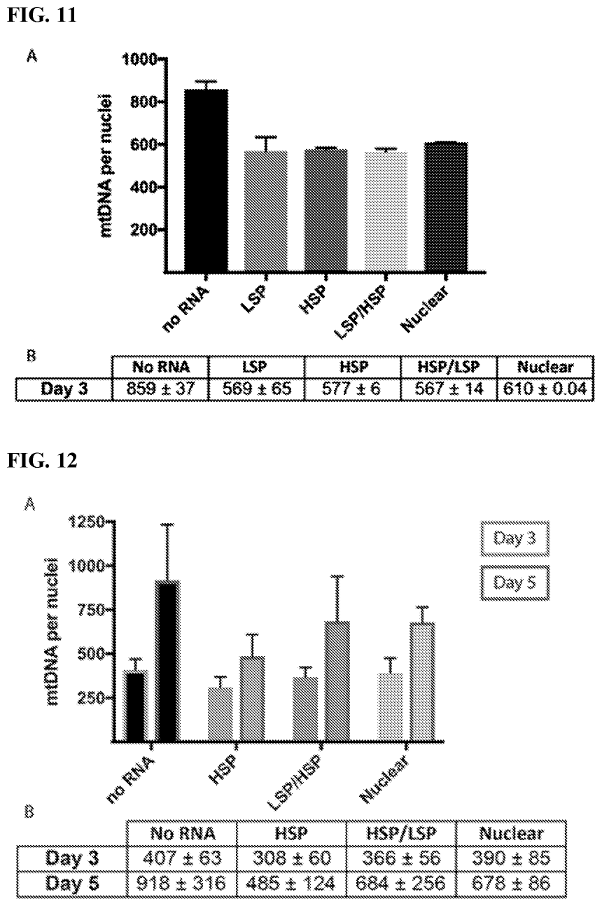

HeLa cells were electroporated with a plasmid encoding mitoCas9, the modified tracrRNA, and respective crRNA targeting either the light strand promoter (LSP), heavy strand promoter (HSP), combination of both HSP and LSP, or a nuclear gene CXCR4. Three days post electroporation, mtDNA were purified and quantified using Taqman multiplex qPCR. The cytochrome B copy number is normalized by β-actin copy to represent a measure of mtDNA content per nuclei. Quantitation of mtDNA content showing depletion of mtDNA in all samples with the crRNA and tracrRNA is illustrated graphically in Panel A of FIG. 11. There was similar mtDNA clearance by targeting the HSP and LSP individually and in combination. There was some off target cleavage since the nuclear target, CXCR4, also demonstrated mtDNA degradation. Values represent mean±SD from 3 biological replicates. A table of values graphed in Panel A of FIG. 11 is presented in Panel B of FIG. 11.

HeLa cells were electroporated with a plasmid expressing mitoCpf1 and the crRNA targeting either HSP alone or in combination with LSP or a nuclear gene target, CXCR4. The mtDNA content normalized to β-actin was surveyed 3 days or 5 days post transfection. A graph illustrating that targeting the HSP sequence yielded the highest depletion of mtDNA is illustrated in Panel A of FIG. 12 (left and right bars in each pair represent day 3 and day 5, respectively). With electroporation, the mixed population of cells resulted in a general expansion of untransfected cells over time leading to a repletion of mtDNA by day 5. However, the HSP sample exhibited less repletion of mtDNA content. A table of values graphed in Panel A of FIG. 12 is presented in Panel B of FIG. 12. Values represent mean±SD from 3 biological replicates.

The sequences of the HSP, LSP, and CXCR4 targets for Cas9 and Cpf1 are presented in Table 3 below. The sequences of primers and probes for detecting the indicated targets are presented in Table 4 below.

TABLE 3

| | |
|---|---|
| AP207_Cas9 | 5′Cy3/mAmAmUmUrUrGrArArArUrCrUrGr |
| LSP crRNA | GrUrUrArGrGrCrGrUrUrUrUrAmGmAmGmC |
| | mUmAmUmGmCmU (SEQ ID NO: 23) |
| AP208_Cas9 | 5′Cy3/mUmAmAmArCrUrGrUrGrGrGrGrGr |
| HSP crRNA | GrUrGrUrCrUrUrGrUrUrUrUrAmGmAmGmC |
| | mUmAmUmGmCmU (SEQ ID NO: 36) |
| AP209_Cas9 | 5′Cy3/mGmAmAmGrCrGrUrGrArUrGrArCr |
| CXCR4 crRNA | ArArArGrArGrGrGrUrUrUrUrAmGmAmGmC |
| | mUmAmUmGmCmU (SEQ ID NO: 37) |
| AP210_Cpf1 | 5′Cy5/mUrArArUmUmUmCmUmAmCrUmCmUm |
| CXCR4 crRNA | UrGmUmAmGmArUrCrArGrArUrArUrArCrA |
| | rCrUrUrCrArGrArUmAmAmC |
| | (SEQ ID NO: 38) |
| AP211_Cpf1 | 5′Cy5/mUrArArUmUmUmCmUmAmCrUmCmUm |
| LSP crRNA | UrGmUmAmGmArUrUrCrUrUrUrUrGrGrCrG |
| | rGrUrArUrGrCrArCmUmUmU |
| | (SEQ ID NO: 22) |
| AP196_Cpf1 | 5′Cy5/mUrArArUmUmUmCmUmAmCrUmCmUm |
| HSP crRNA | UrGmUmAmGmArUrGrGrGrUrUrUrGrGrUrU |
| | rGrGrUrUrCrGrGrGmGmUmA |
| | (SEQ ID NO: 39) |

mX = 2′ OMe
rX = ribonucleotide

TABLE 4

| Target | Primers | Sequence | Probe |
|---|---|---|---|
| Mito | AP212 | GCACAGCCACTTTCC | 5′-FAM/CAAAGAAC |
| LSP | LSP_F | ACACAG | CCTAACACCAGCCTA |
| | | (SEQ ID NO: 40) | ACCAG/Black hole |
| | AP213 | GGGAGTGGGAGGGGA | quencher/-3′ |
| | LSP_R | AAATAATG | (SEQ ID NO: 42) |
| | | (SEQ ID NO: 41) | |
| Mito | AP214 | CATCCTACCCAGCAC | 5′-FAM/CAACCAAA |
| HSP | HSP_F | ACACACAC | CCCCAAAGACACCCC |
| | | (SEQ ID NO: 43) | CCAC/Black hole |
| | AP215 | CGGGGATGCTTGCAT | quencher/-3′ |
| | HSP_R | GTGTAATC | (SEQ ID NO: 45) |
| | | (SEQ ID NO: 44) | |

### Example 4: Therapeutic Nucleic Acid Delivery to Mitochondria

There are over 600 known mtDNA mutations associated with mtDNA diseases, which have diverse clinical features, including maternal inheritance (because mtDNA is inherited strictly from the mother), defects in the central and peripheral nervous systems, muscle defects, and exercise intolerance (Brandon et al., Nucleic Acids Res, 2005, 33 (Database issue), D611-13). Additionally, there are dozens of mtDNA mutations associated with cancer, including bladder, breast, colorectal, gastric, head and neck, lung, ovarian, and prostate cancers (Chatterjee et al., Oncogene, 2006, 23: 4663-74; Hertweck et al., Front. Oncol., 2017, 7:262).

One or more guide RNAs (e.g., sgRNA, or crRNA and tracrRNA) are designed to target a mitochondrial mutation associated with a disease state (e.g., cancer), which are modified for delivery to mitochondria. The one or more guide RNAs will include nucleotide modification (e.g. 2′-OMe modifications) and a cyanine moiety (e.g., Cy3 or Cy5). By targeting a mutant sequence, CRISPR will selectively target pathogenic mutant mtDNA without targeting wildtype mtDNA in the same cell or in other healthy cells.

### Example 5: Cy5-Labeled RNA Delivery to 143B Cell Mitochondria

Cy5 labeled RNA polynucleotides were tested for the ability to localize to the mitochondria in 143B cells, a human osteosarcoma cell line. 143B cells are a culture-based model for examining mitochondiral mtDNA diseases. This particular cell line is relevant for development of therapeutic strategies for mtDNA disease that would involve mitochondrial import of RNA or DNA, such as mitochondrial CRISPR.

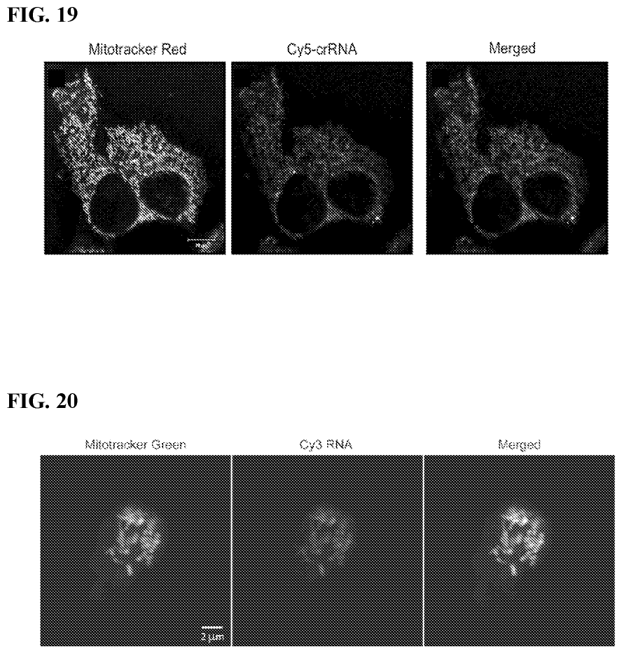

143B cells were transfected with a 41-nucleotide single-stranded RNA polynucleotide. The RNA contained a Cy5 moiety on the 5′-end, and had the following sequence, where “rX” denotes an unmodified RNA nucleotide and “mX” denotes a 2′-O-methyl modified nucleotide: 5′Cy5/mUrArArUmUmUmCmUmAmCrUmCmUmUrGmUmAmGmArUrUrCrUrUrUrUrGrGr CrGrGrUrArUrGrCrArCmUmUmU (SEQ ID NO:22). Cells were transfected via streptolysin O (SLO) using the same conditions as described for the Hela cells shown in FIG. 3B. The cells were imaged at 48 hours post-transfection by confocal microscopy. Representative images of the 143B cells are shown in FIG. 19, with the left panel showing mitochondria stained with Mitotracker Red dye, the center panel showing the Cy5-labeled RNA molecule, and right panel showing merged images. Notably, the merged image shows overlapping signals. Further, Cy5-labeled RNA and Mitotracker Red staining were absent in cellular nuclei, shown as the dark holes in FIG. 19. These results indicate the Cy5-labeled RNA localized to mitochondria in clinically-relevant cells used to develop mtDNA-associated disease therapeutics.

### Example 6: Mitochondrial Localization of Cy3-Labeled RNA in Human Primary Cells

Cy3-labeled RNA polynucleotides were tested for the ability to localize to the mitochondria of human primary cells, which are relevant for clinical translation. Human T cells were isolated from whole blood of an anonymous healthy donor, and transfected with a 36-nucleotide single-stranded RNA polynucleotide. The RNA contained a Cy3 moiety on the 5′-end, and had the following sequence, where “rX” denotes an unmodified RNA nucleotide and “mX” denotes a 2′-O-methyl modified nucleotide: 5′Cy3/mAmAmUmUrUrGrArArArUrCrUrGrGrUrUrArGrGrCrGrUrUrUrUrAmGmAmGmCm UmAmUmGmCmU (SEQ ID NO:23). Primary T cells were transfected by electroporation with the Amaxa Human T Cell Nucleofector Kit according to the manufacturer's protocol, unless otherwise noted. Specifically, 5×10⁶ cells were transfected with 100 nmol of crRNA and cells were subsequently plated into 12-well tissue culture plates. The program of the Nucleofector II was U-014, and the cells were imaged at 48 hours post-transfection by confocal microscopy. Images of a representative cell are illustrated in FIG. 20, with the left panel showing mitochondria stained by Mitotracker Green dye, the center panel showing the Cy3-labeled RNA molecule, and the right panel showing the merged image. The merged image illustrates overlapping signals from the stained mitochondria and Cy3-labeled RNA. These results indicate the Cy3-labeled RNA imported to mitochondria of the cells. Significantly, the results demonstrate feasibility of this approach and high efficiency of Cy3-labeled RNA mitochondrial localization in human primary cells, which is highly relevant to development of therapeutic strategies for mtDNA disease. Further, the mode of delivery via electroporation is clinically relevant, as electroporation is used in clinical trials for ex vivo and in vivo gene delivery.

### REFERENCES

1. Lightowlers R N, Taylor R W, & Turnbull D M (2015) Mutationscausing mitochondrial disease: What is new and what challenges remain? Science 349(6255):1494-1499.
2. Alexeyev M, Shokolenko I, Wilson G, & LeDoux S (2013) Themaintenance of mitochondrial DNA integrity—critical analysis and update. Cold Spring Harb Perspect Biol 5(5):a012641.
3. Kazak L, Reyes A, & Holt I J (2012) Minimizing the damage: repair pathways keep mitochondrial DNA intact. Nat Rev Mol Cell Biol 13(10):659-671.
4. Bacman S R, Williams S L, Pinto M, Peralta S, & Moraes C T (2013) Specific elimination of mutant mitochondrial genomes in patient-derived cells by mitoTALENs. Nat Med 19(9):11111113.
5. Bayona-Bafaluy M P, Blits B, Battersby B J, Shoubridge E A, & Moraes C T (2005) Rapid directional shift of mitochondrial DNA heteroplasmy in animal tissues by a mitochondrially targeted restriction endonuclease. Proc Natl Acad Sci USA 102(40):14392-14397.
6. Gammage P A, Rorbach J, Vincent A I, Rebar E J, & Minczuk M (2014) Mitochondrially targeted ZFNs for selectivedegradation of pathogenic mitochondrial genomes bearing large-scale deletions or point mutations. EMBO Mol Med 6(4):458-466.
7. Hashimoto M, et al. (2015) MitoTALEN: A General Approach toReduce Mutant mtDNA Loads and Restore Oxidative Phosphorylation Function in Mitochondrial Diseases. Mol Ther 23(10):1592-1599.
8. Reddy P, et al. (2015) Selective elimination of mitochondrial mutations in the germline by genome editing. Cell 161(3):459469.
9. Kim H K, et al. (2017) In vivo high-throughput profiling of CRISPR-Cpf1 activity. Nature methods 14(2):153-159.
10. Zetsche B, et al. (2015) Cpf1 is a single RNA-guided endonuclease of a class 2 CRISPR-Cas system. Cell 163(3):759771.
11. Kleinstiver B P, et al. (2016) Genome-wide specificities of CRISPR-Cas Cpf1 nucleases in human cells. Nat Biotechnol 34(8):869-874.
12. Rhee W J & Bao G (2010) Slow non-specific accumulation of 2′deoxy and 2′-O-methyl oligonucleotide probes at mitochondriain live cells. Nucleic Acids Res 38(9):e109.
Brandon M C, Lott M T, Nguyen K C, Spolim S, Navathe S B, Baldi P, Wallace D C: MITOMAP: a human mitochondrial genome database—2004 update. Nucleic Acids Res 2005, 33(Database issue):D611-613.
Chatterjee A, Mambo E, Sidransky D: Mitochondrial DNA mutations in human cancer. Oncogene. vol. 25; 2006: 4663-4674.
Hertweck K L, Dasgupta S: The Landscape of mtDNA Modifications in Cancer: A Tale of Two Cities. Front Oncol 2017, 7:262.

### INFORMAL SEQUENCE LISTING

In the following, “mX” designates a nucleotide having a 2′OMe modification, and “rX” designates a ribonucleotide.

SEQ ID NO: 1
5′Cy3/mUmGmGrGrGrGrGrUrGrUrCrUrUrUrGrGrGrGrUrUrG
rUrUrUrUrArGrArGrCrUrArUrGrCrUrGrUrUmUmUmG
SEQ ID NO: 2
5′Cy3/mUmAmArUrUrUrCrUrArCrUrCrUrUrGrUrArGrArUrG
rGrGrUrUrUrGrGrUrUrGrGrUrUrCrGmGmGmG
SEQ ID NO: 3
rUrArArUrUrUrCrUrArCrUrCrUrUrGrUrArGrArUrGrGrGrU
rUrUrGrGrUrUrGrGrUrUrCrGmGmGmG/3′Cy3
SEQ ID NO: 4
mUrArArUmUmUmCmUmAmCrUmCmUmUrGmUmAmGmArUrGrGrGrU
rUrUrGrGrUrUrGrGrUrUrCrGrGmGmG/3′Cy3
SEQ ID NO: 5
mUrArArUmUmUmCmUmAmCrUmCmUmUrGmUmAmGmArUrGrGrGrU
rUrUrGrGrUrUrGrGrUrUrCrGrGrGmGmUmA/3′Cy3
SEQ ID NO: 6
5′Cy3/mGmAmGmUrGrGrUrUrArGrUrUrUrUrArUrUrArGrGrG
rUrUrUrUrAmGmAmGmCmUmAmUmGmCmU
SEQ ID NO: 7
mA*mG*mCmAmUmAmGmCmAAGUUAAAAUAAGGCUAGUCCGUUmAmU
mCmAmAmCmUmUmGmAmAmAmAmAmGmUmGmGmCmAmCmCmGmAmGmU
mCmGmGmUmGmCmU*mU*mU
(IDT tracrRNA with 5′ Cy5; “*” designates
phosphorothioate bond)
SEQ ID NO: 8
5′Cy5/TAATTTCTACTCTTGTAGATGGGTTTGGTTGGTTCGGGGT
A/3′Cy3
SEQ ID NO: 9
5′Cy5/mAmGmCmAmUmAmGmCmArArGrUrUrArArArArUrArArG
rGrCrUrArGrUrCrCrGrUrUrArUrCrArAmCmUmUmGmAmAmAmA
mAmGmUmGmGmCmAmCmCmGmAmGmUmCmGmGmUmGmCmUmUmU
SEQ ID NO: 10
5′Cy5/mUrArArUmUmUmCmUmAmCrUmCmUmUrGmUmAmGmArUrG
rGrGrUrUrUrGrGrUrUrGrGrUrUrCrGrGrGmGmUmA
SEQ ID NO: 11
5′Cy5/mGmUmCmAmAmAmAmGmAmCmCmUmUmUmUmUrArArUmUmU
mCmUmAmCrUmCmUmUrGmUmAmGmArUrGrGrGrUrUrUrGrGrUrU
rGrGrUrUrCrGrGrGmGmUmA
SEQ ID NO: 12
5′Cy3/TAATTTCTACTCTTGTAGATGGGTTTGGTTGGTTCGGGGTA
SEQ ID NO: 13
TAATTTCTACTCTTGTAGATGGGTTTGGTTGGTTCGGGGTA/3′Cy3
SEQ ID NO: 14
5′Cy5/AGCATAGCAAGTTAAAATAAGGCTAGTCCGTTATCAACTTGA
AAAAGTGGCACCGAGTCGGTGCTTT
SEQ ID NO: 15
5′ATTO647n/TAATTTCTACTCTTGTAGATGGGTTTGGTTGGTTCGG
GGTA
SEQ ID NO: 16
TAATTTCTACTCTTGTAGATGGGTTTGGTTGGTTCGGGGTA/3′Cy3
SEQ ID NO: 17
5AmMC6/TAATTTCTACTCTTGTAGATGGGTTTGGTTGGTTCGGGGT
A/3′Cy3
SEQ ID NO: 183
5dSp/TAATTTCTACTCTTGTAGATGGGTTTGGTTGGTTCGGGGT
A/3′Cy3
SEQ ID NO: 20
5′Cy5/GCTGCTAACCCCATACCCCGAACCAACCAAACCCCAAAGACA
ATGGGCAAGCCCATCCCCAACCCCTTGCTTGGCTTGGACAGCACCTAA
SEQ ID NO: 21
5′Cy3/TTAGGTGCTGTCCAAGCCAAGCAAGGGGTTGGGGATGGGCTT
GCCCATTGTCTTTGGGGTTTGGTTGGTTCGGGGTATGGGGTTAGCAGC

### EMBODIMENTS

The present disclosure further provides the following embodiments:

Embodiment 1. A compound comprising a polynucleotide covalently linked to a cyanine moiety, wherein the polynucleotide comprises a nucleotide sequence that is fully complementary to a nucleotide sequence of a mitochondrial polynucleotide.

Embodiment 2. The compound of Embodiment 1, wherein the mitochondrial polynucleotide is a mitochondrial DNA or a mitochondrial RNA.

Embodiment 3. The compound of Embodiment 1, wherein the polynucleotide comprises a one or more ribonucleotides, one or more deoxyribonucleotides, and/or one or more 2′-modified nucleotides.

Embodiment 4. The compound of Embodiment 3, wherein the one or more 2′-modified nucleotides are 2′-amine modified nucleotides, 2′-O-methyl modified nucleotides or any combination thereof.

Embodiment 5. The compound of any one of Embodiments 1-4, wherein the cyanine moiety is attached at the 5′-end of the polynucleotide.

Embodiment 6. A compound comprising a polynucleotide covalently linked to a cyanine moiety, wherein the cyanine moiety is attached at the 5′-end of the polynucleotide, and wherein the polynucleotide comprises one or more ribonucleotides.

Embodiment 7. The compound of any one of Embodiments 1-6, wherein the cyanine moiety is a streptocyanine moiety, a hemicyanine moiety, or a closed cyanine moiety.

Embodiment 8. The compound of any one of Embodiments 1-7, wherein the cyanine moiety is fluorescent.

Embodiment 9. The compound of any one of Embodiments 1-7, wherein the cyanine moiety is not fluorescent.

Embodiment 10. The compound of any one of Embodiments 1-7, wherein said cyanine moiety is a Cy2 moiety, Cy3 moiety, Cy3B moiety, Cy3.5 moiety, Cy5 moiety, Cy5.5 moiety, Cy7.5 moiety, or Cy7 moiety.

Embodiment 11. The compound of any one of Embodiments 1-10, wherein the polynucleotide comprises one or more 2′-modified nucleotides.

Embodiment 12. The compound of Embodiment 11, wherein the one or more 2′-modified nucleotides comprise a 2′-amine modified nucleotide, a 2′-O-methyl modified nucleotide, or any combination thereof.

Embodiment 13. The compound of any one of Embodiments 1-10, wherein the polynucleotide is a polyribonucleotide.

Embodiment 14. The compound of any one of Embodiments 1-13, further comprising another cyanine moiety attached at the 3′-end of the polynucleotide.

Embodiment 15. The compound of any one of Embodiments 1-14, wherein the polynucleotide is about 10-200 nucleotides in length.

Embodiment 16. The compound of any one of Embodiments 1-15, wherein the polynucleotide is CRISPR/Cas9 single-guide RNA, an RNA interference polynucleotide, or an antisense oligonucleotide.

Embodiment 17. A cell comprising the compound of any one of Embodiments 1-16.

Embodiment 18. A method of delivering an polynucleotide into mitochondria of a cell, the method comprising contacting said cell with the compound of any one of Embodiments 1-16.

Embodiment 19. A complex comprising the compound of any one of Embodiments 1-16 and an RNA-guided protein.

Embodiment 20. The complex of Embodiment 19, wherein the RNA-guided protein is an RNA-guided enzyme.

Embodiment 21. The complex of Embodiment 20, wherein the RNA-guided enzyme is an RNA-guided endonuclease enzyme.

Embodiment 22. The complex of Embodiment 19, wherein said RNA-guided endonuclease enzyme comprises a mitochondrial localization amino acid sequence covalently attached to N-terminus of said RNA-guided endonuclease enzyme.

Embodiment 23. The complex of Embodiment 21 or 22, wherein said RNA-guided endonuclease enzyme is Cas9, Cpf1, a Class II CRISPR endonuclease or a variant thereof.

Embodiment 24. A method of altering the sequence or the expression of at least one mitochondrial polynucleotide, the method comprising introducing into an eukaryotic cell the compound of any one of Embodiments 1 to 16 or the complex of any one of Embodiments 19 to 23.

Embodiment 25. The method of Embodiment 24, comprising introducing into said eukaryotic cell an RNA-guided endonuclease enzyme.

Embodiment 26. The method of Embodiment 25, wherein said RNA-guided endonuclease enzyme comprises a mitochondrial localization amino acid sequence covalently attached to N-terminus of said RNA-guided endonuclease enzyme.

Embodiment 27. The method of Embodiment 25, wherein said RNA-guided endonuclease enzyme is Cas9, Cpf1, a Class II CRISPR endonuclease or a variant thereof.

Embodiment 28. The method of Embodiment 25, wherein said RNA-guided endonuclease enzyme is a base-editor.

Embodiment 29. A method of treating a mitochondrial disorder in a subject in need thereof, the method comprising administering to said subject the compound of any one of Embodiments 1 to 16 or the complex of any one of Embodiments 19 to 23.

Embodiment 30. The method of Embodiment 29, comprising introducing into said eukaryotic cell an RNA-guided endonuclease enzyme.

Embodiment 31. The method of Embodiment 30, wherein said RNA-guided endonuclease enzyme comprises a mitochondrial localization amino acid sequence covalently attached to N-terminus of said RNA-guided endonuclease enzyme.

Embodiment 32. The method of Embodiment 30, wherein said RNA-guided endonuclease enzyme is Cas9, Cpf1, a Class II CRISPR endonuclease or a variant thereof.

Embodiment 33. The method of Embodiment 29, wherein said mitochondrial disorder is myoclonic epilepsy with ragged red fibers (MERRF); mitochondrial myopathy, encephalopathy, lactacidosis, and stroke (MELAS); maternally inherited diabetes and deafness (MIDD); Leber's hereditary optic neuropathy (LHON); chronic progressive external ophthalmoplegia (CPEO); Leigh disease; Kearns-Sayre syndrome (KSS); Friedreich's Ataxia (FRDA); co-enzyme QlO (CoQlO) deficiency; complex I deficiency; complex II deficiency; complex III deficiency; complex IV deficiency; complex V deficiency; myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; or cancer.

What is claimed is:

1. A compound comprising a polynucleotide covalently linked to a cyanine moiety, wherein the polynucleotide comprises a nucleotide sequence that is fully complementary to a nucleotide sequence of a mitochondrial polynucleotide.

2. The compound of claim 1, wherein the mitochondrial polynucleotide is a mitochondrial DNA or a mitochondrial RNA.

3. The compound of claim 1, wherein the polynucleotide comprises a one or more ribonucleotides, one or more deoxyribonucleotides, and/or one or more 2′-modified nucleotides.

4. The compound of claim 3, wherein the one or more 2′-modified nucleotides are 2′-amine modified nucleotides, 2′-O-methyl modified nucleotides or any combination thereof.

5. The compound of any one of claims 1-4, wherein the cyanine moiety is attached at the 5′-end of the polynucleotide.

6. A compound comprising a polynucleotide covalently linked to a cyanine moiety, wherein the cyanine moiety is attached at the 5′-end of the polynucleotide, and wherein the polynucleotide comprises one or more ribonucleotides.

7. The compound of any one of claim 1-4 or 6, wherein the cyanine moiety is a streptocyanine moiety, a hemicyanine moiety, or a closed cyanine moiety.

8. The compound of any one of claim 1-4 or 6, wherein the cyanine moiety is fluorescent.

9. The compound of any one of claim 1-4 or 6, wherein the cyanine moiety is not fluorescent.

10. The compound of any one of claim 1-4 or 6, wherein said cyanine moiety is a Cy2 moiety, Cy3 moiety, Cy3B moiety, Cy3.5 moiety, Cy5 moiety, Cy5.5 moiety, Cy7.5 moiety, or Cy7 moiety.

11. The compound of any one of claim 1-4 or 6, wherein the polynucleotide comprises one or more 2′-modified nucleotides.

12. The compound of claim 11, wherein the one or more 2′-modified nucleotides comprise a 2′-amine modified nucleotide, a 2′-O-methyl modified nucleotide, or any combination thereof.

13. The compound of any one of claim 1-4 or 6, wherein the polynucleotide is a polyribonucleotide.

14. The compound of any one of claim 1-4 or 6, further comprising another cyanine moiety attached at the 3′-end of the polynucleotide.

15. The compound of any one of claim 1-4 or 6, wherein the polynucleotide is about 10-200 nucleotides in length.

16. The compound of any one of claim 1-4 or 6, wherein the polynucleotide is CRISPR/Cas9 single-guide RNA, an RNA interference polynucleotide, or an antisense oligonucleotide.

17. A cell comprising the compound of any one of claim 1-4 or 6.

18. A method of delivering a polynucleotide into mitochondria of a cell, the method comprising contacting said cell with the compound of any one of claim 1-4 or 6.

19. A complex comprising the compound of any one of claim 1-4 or 6, and an RNA-guided protein.

20. The complex of claim 19, wherein the RNA-guided protein is an RNA-guided enzyme.

21. The complex of claim 20, wherein the RNA-guided enzyme is an RNA-guided endonuclease enzyme.

22. The complex of claim 19, wherein said RNA-guided endonuclease enzyme comprises a mitochondrial localization amino acid sequence covalently attached to N-terminus of said RNA-guided endonuclease enzyme.

23. The complex of claim 21, wherein said RNA-guided endonuclease enzyme is Cas9, Cpf1, a Class II CRISPR endonuclease or a variant thereof.

24. A method of altering the sequence or the expression of at least one mitochondrial polynucleotide, the method comprising introducing into an eukaryotic cell the compound of any one of claim 1-4 or 6, or a complex comprising said compound and an RNA-guided protein.

25. The method of claim 24, comprising introducing into said eukaryotic cell an RNA-guided endonuclease enzyme.

26. The method of claim 25, wherein said RNA-guided endonuclease enzyme comprises a mitochondrial localization amino acid sequence covalently attached to N-terminus of said RNA-guided endonuclease enzyme.

27. The method of claim 25, wherein said RNA-guided endonuclease enzyme is Cas9, Cpf1, a Class II CRISPR endonuclease or a variant thereof.

28. The method of claim 25, wherein said RNA-guided endonuclease enzyme is a base-editor.

29. A method of treating a mitochondrial disorder in a subject in need thereof, the method comprising administering to said subject the compound of any one of claim 1-4 or 6, or a complex comprising said compound and an RNA-guided protein.

30. The method of claim 29, comprising introducing into said eukaryotic cell an RNA-guided endonuclease enzyme.

31. The method of claim 30, wherein said RNA-guided endonuclease enzyme comprises a mitochondrial localization amino acid sequence covalently attached to N-terminus of said RNA-guided endonuclease enzyme.

32. The method of claim 30, wherein said RNA-guided endonuclease enzyme is Cas9, Cpf1, a Class II CRISPR endonuclease or a variant thereof.

33. The method of claim 29, wherein said mitochondrial disorder is myoclonic epilepsy with ragged red fibers (MERRF); mitochondrial myopathy, encephalopathy, lactacidosis, and stroke (MELAS); maternally inherited diabetes and deafness (MIDD); Leber's hereditary optic neuropathy (LHON); chronic progressive external ophthalmoplegia (CPEO); Leigh disease; Kearns-Sayre syndrome (KSS); Friedreich's Ataxia (FRDA); co-enzyme QlO (CoQlO) deficiency; complex I deficiency; complex II deficiency; complex III deficiency; complex IV deficiency; complex V deficiency; myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; or cancer.
